(19) United States
(12) Patent Application Publication
(10) Pub. No.: US 2021/0113536 A1
(43) Pub. Date: Apr. 22, 2021

(54) METHODS AND COMPOSITIONS FOR DETECTING AND TREATING VENOUS THROMBOEMBOLISM

(71) Applicant: United States Government As Represented By The Department of Veteran Affairs, Washington, DC (US)

(72) Inventors: Pradeep Natarajan, Boston, MA (US); Derek Klarin, Gainesville, FL (US); Scott Damrauer, Philadelphia, PA (US)

(21) Appl. No.: 17/075,101

(22) Filed: Oct. 20, 2020

Related U.S. Application Data
(60) Provisional application No. 63/037,375, filed on Jun. 10, 2020.
(60) Provisional application No. 62/923,898, filed on Oct. 21, 2019.

(51) Int. Cl. A61K 31/443; A61K 45/06; C12Q 1/6883

(52) U.S. Cl. CPC A61K 31/443; A61K 45/06; C12Q 2600/118; C12Q 2600/156; C12Q 1/6883

(57) ABSTRACT

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor. Disclosed are methods of diagnosing and VTE, the method comprising diagnosing the subject as being at greater risk of developing VTE; and administering a venous thrombus size lowering therapy to the subject, wherein the venous thrombus size lowering therapy is a PAI-1 inhibitor. Disclosed are methods of determining a PRS for developing VTE in a subject comprising identifying the presence of one or more of the 297 SNPs identified in FIG. 19 are present in a biological sample from the subject; and calculating the PRS by summing the weighted risk score associated with each SNP identified.

### CROSS-REFERENCE TO RELATED APPLICATIONS

This application claims the benefit of U.S. Provisional Patent Application No. 62/923,898, filed on Oct. 21, 2019 and U.S. Provisional Patent Application No. 63/037,375, filed on Jun. 10, 2020, each of which is incorporated by reference herein in its entirety.

### STATEMENT REGARDING FEDERALLY SPONSORED RESEARCH

This invention was made with government support under Grant Numbers K08HL140203 and R01HL142711 awarded by the National Institutes of Health, and Grant Numbers I01-01BX03340, I01-BX003362, and I01-CX001025 awarded by the Department of Veterans Affairs. The government has certain rights in this invention.

### BACKGROUND

Venous thromboembolism (VTE) is a significant cause of mortality, yet its genetic determinants remain incompletely defined. VTE is a complex disease impacted by both environmental and genetic determinants, and the narrow-sense heritability of VTE has been estimated to be approximately 30%. At the time of the current analysis, genome-wide association studies (GWAS) revealed only 11 loci reaching genome-wide significance, leaving a significant portion of VTE heritability unknown.

The data herein provides new mechanistic insights into the genetic epidemiology of VTE and indicates a greater overlap among venous and arterial cardiovascular disease than previously known.

### BRIEF SUMMARY

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor.

Disclosed are methods of diagnosing and treating venous thromboembolism (VTE), the method comprising diagnosing the subject as being at greater risk of developing VTE; and administering a venous thrombus size lowering therapy to the subject, wherein the venous thrombus size lowering therapy is a PAI-1 inhibitor.

Disclosed are methods of determining a PRS for developing VTE in a subject comprising identifying the presence of one or more of the 297 SNPs identified in FIG. 19 are present in a biological sample from the subject; and calculating the PRS by summing the weighted risk score associated with each SNP identified.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a venous thromboembolic disease genetic discovery and replication study design. Abbreviations: MVP, Million Veteran Program; VTE, Venous thromboembolism; PCs, Principal Components

FIGS. 2A, 2B, and 2C show blood lipids and VTE risk. Association of the 222 variant lipid genetic risk score with VTE in a multivariable Mendelian randomization analysis. Logistic regression odds ratios are displayed per 1-standard deviation genetically increased A) LDL cholesterol, B) HDL cholesterol, and C) triglycerides. Wald statistic two-sided values of P are displayed. Summary-level lipids data from up to 319,677 participants of the Global Lipids Genetics Consortium, and VTE association data from MVP (N=8,929 cases; 181,337 controls) and UK Biobank (N=14,222 cases; 372,102 controls) were used for this analysis. Gray boxes reflect the inverse-variance weight for each study. Abbreviations: HDL, High-Density Lipoprotein; LDL, Low-Density Lipoprotein; MVP, Million Veteran Program; UKB, UK Biobank

FIG. 3 shows a functional assessment of PAI-1 in murine models. Abbreviations: PAI-1, Plasminogen Activator Inhibitor-1; Tg, Transgenic; WT, Wild Type

FIGS. 4A and 4B show a genome-wide polygenic risk score for VTE. A) Distribution of the PRSVTE in the MVP release 3.0 dataset (n=55,965). The x-axis represents the PRS with values transformed to have a mean of 0 and standard deviation of 1. The region shaded in blue represents those with the highest 5% of PRSVTE values. B) VTE odds ratios in MVP release 3.0 data for carriers of the F5 p.R506Q and F2 G20210A mutations. In addition, the odds ratio for individuals with the highest 5% PRSVTE compared to individuals among the lower 95% of PRSVTE, as well as for carriers of the F5 p.R506Q and F2 G20210A mutations within the highest 5% PRSVTE are depicted. Wald statistic two-sided values of P are displayed. Abbreviations: VTE, Venous Thromboembolism; PRS, Polygenic Risk Score; Chr, Chromosome; MVP, Million Veteran Program; CI, Confidence Interval

FIG. 5 shows a genome-wide polygenic risk score and incident VTE events. Hazard ratios calculated from the Cox Proportional hazards model for incident VTE events in the Women's Health Initiative study for carriers of the F5 p.R506Q and F2 G20210A mutations. The hazard ratio for individuals with the highest 5% PRSVTE compared to individuals among the lower 95% of PRSVTE is also depicted. Two-sided values of P are displayed. Abbreviations: VTE, Venous Thromboembolism; PRS, Polygenic Risk Score; Chr, Chromosome; CI, Confidence Interval.

FIG. 6 shows an overall study design of a genome-wide association study to identify novel VTE risk variants. Abbreviations: PAI-1, Plasminogen Activator Inhibitor-1; BMI, Body-Mass Index; CAD, Coronary Artery Disease; GLGC, Global Lipids Genetics Consortium; GTEx, Genotype-Tissue Expression Project; LAS, Large Artery Stroke; MVP, Million Veteran Program; PAD, Peripheral Artery Disease; PheWAS, Phenome-wide Association Study; VTE, Venous Thromboembolism; WHI, Women's Health Initiative

FIG. 7 shows a quantile-quantile plot for the discovery VTE GWAS in MVP (N=11,844 VTE cases and 251,951 controls). The expected logistic regression association P values versus the observed distribution of P values for VTE association (Wald statistic) are displayed. Abbreviations: GWAS, Genome-wide Association Study; MVP, Million Veteran Program; VTE, Venous Thromboembolism

FIG. 8 shows a quantile-quantile plot for the discovery VTE GWAS in UK Biobank (N=14,222 VTE cases and 372,102 controls). The expected logistic regression association P versus the observed distribution of P values for VTE association (Wald statistic) are displayed. All P values were two-sided. Abbreviations: GWAS, Genome-wide Association Study; VTE, Venous Thromboembolism

FIG. 9 shows a quantile-quantile plot for the trans-ethnic VTE GWAS meta-analysis in MVP and UK Biobank (N=26,066 VTE cases and 624,053 controls).

FIG. 10 shows a Manhattan plot for the trans-ethnic VTE GWAS (N=26,066 VTE cases and 624,053 controls). Plot of −log 10(P) for association (logistic regression Wald statistic) of genotyped and imputed variants by chromosomal position (alternating blue and yellow) for all autosomal polymorphisms analyzed in the UK Biobank and MVP VTE GWAS meta-analysis. Logistic regression two-sided P values are displayed.

FIG. 11 shows a LocusCompare visualization of colocalization between ZFPM2 VTE GWAS and PAI-1 pQTL signals. Colocalization between the ZFPM2 locus in the VTE GWAS (N=23,151 VTE cases, 553,439 controls) and PAI-1 human plasma pQTL (N=3,301) signals. Two-sided values of P are displayed.

FIG. 12 shows a genetic correlation of VTE with atherosclerosis in different arterial beds. All values of P are two-sided; genetic correlations with associated standard errors are displayed. Abbreviations: VTE, Venous Thromboembolism; CAD, Coronary Artery Disease; LAS, Large Artery Stroke; PAD, Peripheral Artery Disease

FIG. 13 is a table showing Logistic Regression (Wald statistic) odds ratios and two-sided P values for 11 previously identified genome-wide significant VTE loci in MVP+UKBB GWAS analysis (N=26,066 VTE cases; 624,053 controls). * Genes for variants that are outside the transcript boundary of a protein-coding gene are shown with nearest candidate gene in parentheses [eg, (CD93)]. Abbreviations: EA, Effect Allele; NEA, Non Effect Allele; EAF, Effect Allele Frequency; OR, Odds Ratio; AFR, African Ancestry; EUR, European Ancestry; HIS, Hispanic Ancestry; UKB, UK Biobank.

FIG. 14 is a table showing Logistic Regression (Wald statistic) odds ratios and two-sided P values for 28 candidate novel genome-wide significant VTE loci identified in the MVP+UKBB genome-wide association study (N=26,066 VTE cases; 624,053 controls). * Genes for variants that are outside the transcript boundary of a protein-coding gene are shown with nearest candidate gene in parentheses [eg, (EPHA3)]. Abbreviations: MVP, Million Veteran Program; EA, Effect Allele; NEA, Non Effect Allele; EAF, Effect Allele Frequency; OR, Odds Ratio; AFR, African Ancestry; EUR, European Ancestry; HIS, Hispanic Ancestry; UKB, UK Biobank

FIG. 15 is a table showing Logistic Regression (Wald statistic) odds ratios and two-sided P values for 22 successfully replicated novel genome-wide significant VTE loci [(Discovery N=26,066 VTE cases; 624,053 controls), (Replication N=17,672 VTE cases; 167,295 controls)]

FIG. 16 is a table showing Logistic Regression (Wald statistic) odds ratios and two-sided P values for 6 unsuccessfully replicated novel genome-wide significant VTE loci [(Discovery N=26,066 VTE cases; 624,053 controls), (Replication N=17,288 VTE cases; 166,914 controls)]

FIG. 17 is a table showing linear regression effect estimates, standard errors, and two-sided P values for 222 variants (across 222 distinct loci) used for weighted genetic risk score. Effect estimates/P values are taken from 2017 GLGC exome array analysis. * Variants were included only in the MVP genetic risk score, as these variants did not pass quality control in UK Biobank (MAF <0.003). Abbreviations: EA, Effect Allele; NEA, Non-effect Allele; EAF, Effect Allele Frequency; SE, Standard Error; GLGC, Global Lipids Genetics Consortium; HDL-C, High-Density Lipoprotein Cholesterol; LDL-C, Low-Density Lipoprotein Choleterol; TG, Triglycerides

FIG. 18 is a table showing genome-wide significant ZFPM2-PAI-1 linear regression pQTL associations in human plasma from the INTERVAL study (N=3,301) for 99.99% credible set variants at the ZFPM2 locus. Two-sided values of P are displayed.

FIG. 19 is a table showing logistic regression association results (Wald statistic) for 297 variants used for weighted, VTE polygenic risk score (PRSVTE). Two-sided values of P are displayed.

FIG. 20 is a table showing Logistic Regression (Wald statistic) odds ratios and two-sided P values for 33 genome-wide significant VTE loci in MVP stratified by ethnicity (African N=2,261 VTE cases, 49,400 controls; European N=8,929 cases, 181,337 controls; Hispanic N=654 VTE cases, 21,214 controls). * Genes for variants that are outside the transcript boundary of a protein-coding gene are shown with nearest candidate gene in parentheses [eg, (CD93)]

FIG. 21 is a table showing Logistic Regression (Wald statistic) odds ratios and two-sided P values for 3 previously reported African specific variants in African ancestry MVP participants (African N=2,261 VTE cases, 49,400 controls)

FIG. 22 is a table showing Logistic regression effect estimate and two-sided P value (Wald statistic) for 15 additional independent genome-wide VTE variants identified with GCTA-COJO software (23,151 VTE cases, 553,439 controls of European ancestry). Abbreviations: EA, Effect Allele; NEA, Non-effect Allele; EAF, Effect Allele Frequency; SE, Standard Error; LD Correlation, LD correlation between the lead variant (i) and variant i+1 for the variants on the list.

FIG. 23 is a table showing Logistic regression effect estimate (Wald statistic) and two-sided P values for statistically significant (P<1.1×10⁻⁶) phenome-wide association results for lead VTE DNA sequence variants and the PRSVTE

FIG. 24 is a table showing PAD, CAD, and LAS logistic regression association statistics for 30 autosomal genome-wide significant VTE risk loci in the MVP, CARDIoGRAMplusC4D, and MEGASTROKE analyses, respectively. Two-sided values of P (Wald statistic) are displayed. Abbreviations: EAF, Effect Allele Frequency; PAD, Peripheral Artery Disease; CAD, Coronary Artery Disease; LAS, Large Artery Stroke; SE, Standard Error

FIG. 25 is a table showing Genome-wide significant linear regression pQTL associations in human plasma from the INTERVAL study (N=3,301) aligned to the VTE risk allele. Two-sided values of P are displayed.

FIG. 26 is a table showing Genome-wide significant linear regression pQTL associations in human plasma from the INTERVAL study (N=3,301) directly involved in the coagulation cascade, aligned to the VTE risk allele. Two-sided values of P are displayed.

FIG. 27 is a table showing a summary of 99.99% credible sets for 12 VTE loci with 6 or fewer VTE associated variants from the MR-MEGA fine-mapping analysis (N=26,066 VTE cases; 624,053 controls)

FIG. 28 is a table showing VTE logistic Regression association results (Wald statistic) for the Factor 5 Leiden mutation, Prothrombin gene (Factor 2) mutation, and PRSVTE stratified by sex in MVP release 3.0 data. Two-sided values of P are displayed.

FIG. 29 is a table showing hazard ratios (derived from Cox proportional hazard models) for incident VTE events in the Women's Health Initiative study stratified by sub-study. Two sided values of P are displayed. *F5 p.R506Q and F2 G20210A effect estimates are aligned to the minor allele

FIG. 30 is a table showing PRSVTE hazard ratios (derived from Cox proportional hazard models) for incident VTE events in the Women's Health Initiative study stratified by hormone replacement therapy use. Two-sided values of P are displayed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a single nucleotide polymorphism” includes a plurality of such single nucleotide polymorphisms, reference to “the PAI-1 inhibitor” is a reference to one or more PAI-1 inhibitors and equivalents thereof known to those skilled in the art, and so forth.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

By “treat” is meant to administer a therapeutic, such as a venous thrombus size lowering therapy, to a subject, such as a human or other mammal (for example, an animal model), that has an increased susceptibility for developing venous thromboembolism (VTE), in order to prevent or delay a worsening of the effects of the disease or condition, or to partially or fully reverse the effects of the disease or condition (e.g. VTE).

By “prevent” is meant to minimize the chance that a subject who has an increased susceptibility for developing VTE will develop VTE.

As used herein, the terms “administering” and “administration” refer to any method of providing a therapeutic, such as a venous thrombus size lowering therapy (e.g., PAI-1), to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject or induce apoptosis.

As used herein, “biological sample” refers to any sample that can be from or derived from a mammal, particularly a human patient, e.g., bodily fluids (blood, saliva, urine etc.), biopsy, tissue, and/or waste from the patient. Thus, tissue biopsies, stool, sputum, saliva, blood, plasma, serum, lymph, tears, sweat, urine, vaginal secretions, or the like can easily be screened for SNPs, as can essentially any tissue of interest that contains the appropriate nucleic acids. These samples are typically taken, following informed consent, from a patient by standard medical laboratory methods. The sample may be in a form taken directly from the patient, or may be at least partially processed (purified) to remove at least some non-nucleic acid material.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The “polygenic risk score” is used to define an individuals' risk of developing a disease or progressing to a more advanced stage of a disease, based on a large number, typically hundreds or thousands, of common genetic variants each of which might have modest individual effect sizes contribute to the disease or its progression, but in aggregate have significant predicting value. In the present case, the polygenic risk score is used to predict the likelihood that a patient will develop VTE using single nucleotide polymorphisms (SNPs) associated with VTE. The log of the odds ratio (OR) from every variant reaching a P<0.1 in the discovery dataset may be used to calculate the polygenic risk score. Specifically, for each variant used in the score, the log of the Odds Ratio for each variant is multiplied by the number of reference alleles (0, 1 or 2) carried by the individual. The resulting log-additive score is then standardized to the same measure in population controls by the same measurement amongst population controls, resulting in the final polygenic risk score. In some aspects, a P<1×10⁻⁵ can be used.

By “probe,” “primer,” or oligonucleotide, it is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for a SNP can have at least 80%-90% sequence complementarity, preferably at least 91%-95% sequence complementarity, more preferably at least 96%-99% sequence complementarity, and most preferably 100% sequence complementarity to the sequence surrounding the SNP to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, or electrophoretic mobility shift assay (EMSA).

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Treating

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor. Disclosed are methods of treating venous thromboembolism (VTE) in a subject in need thereof, the method comprising administering a venous thrombus size lowering therapy to the subject, wherein the venous thrombus size lowering therapy is a plasminogen activator inhibitor 1 (PAI-1) inhibitor.

Disclosed are methods of treating a subject at risk for VTE comprising administering a PAI-1 inhibitor to the subject. In some aspects, the subject was previously determined to be at risk for VTE.

In some aspects, the PAI-1 inhibitor reduces venous thrombus size in the subject by increasing the conversion of plasminogen to plasmin and increasing the interaction of circulating monocytes with the glycoprotein vitronectin within the thrombus and adjacent vein wall, thereby increasing thrombus clearance and reducing the risk of VTE in the subject.

In some aspects, a PAI-1 inhibitor can be, but is not limited to, vorapaxar, tiplaxtinin (PAI-039), tiplatinin, TM5275, TVASSS, TVAVIS, TM5001, TM5007, TM5275, XR334, XR330, XR1853, XR5082, XR5118, XR11211, XR5967, AR-H029953XX, WAY-140312, HP129, XR1853, XR5118, diaplasinin (PAI-749), 535225, XK4044, T-1776Na, tannic acid, gallic acid, CDE-066, CDE-082, CDE-096, AZ3976, embelin, and bis-ANS. Additional PAI-1 inhibitors can be those listed in A. Rouch et al., “Small molecules inhibitors of plasminogen activator inhibitor-1—An overview”, European Journal of Medicinal Chemistry 92 (2015) 619-636, which is incorporated by reference in its entirety.

In some aspects, the subject has been determined to be at a greater risk of developing VTE. In some aspects, a subject has previously been determined to be at a greater risk of developing VTE by a physician. In some aspects, a greater risk of developing VTE is determined based on family history or a genetic predisposition. In some aspects, a greater risk of developing VTE is based on whether the subject has cancer, is having or has recently had major surgery. In some aspects, the subject has been identified to have a polygenic risk score that corresponds to a high risk group. In some aspects, a high PRS indicates a higher risk for developing VTE. For example, a PRS within the top 10, 9, 8, 7, 6, 5, 4, 3, 2, 1, 0.5% of the population indicates the subject is at greater risk of developing VTE.

In some aspects, the biological sample can be any sample from the subject. For example, the biological sample can be, but is not limited to, blood, plasma, serum, urine, spinal fluid, sputum, cells, and tissue.

In some aspects, the subject can be a human. In some aspects, the subject can be a mammal.

In some aspects, the methods can further comprise administering a second therapy to the subject. In some aspects, the second therapy can be an LDL lowering therapy. In some aspects, the LDL lowering therapy can be a statin or a PCSK9 inhibitor. In some aspects, the second therapy can be an anticoagulant or a thrombolytic agent. In some aspects, an anticoagulant can be, but is not limited to, coumadin, dabigatran, rivaroxaban, apixaban. In some aspects, the second therapy can be a heparinoid therapy.

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor wherein the subject has been diagnosed as being at risk of developing VTE by a method comprising identifying the presence of a F5 Leiden pR506Q or a prothrombin G202010A single nucleotide polymorphism (SNP) present in a biological sample from the subject.

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor wherein the subject has been identified to have a F5 Leiden pR506Q or a prothrombin G202010A single nucleotide polymorphism (SNP) by a method comprising obtaining a biological sample from the subject; and detecting whether a F5 Leiden pR506Q or a prothrombin G202010A single nucleotide polymorphism (SNP) is present in the biological sample by contacting the sample with primer or probe specific to a F5 Leiden pR506Q or a prothrombin G202010A single nucleotide polymorphism (SNP) and detecting binding between a F5 Leiden pR506Q or a prothrombin G202010A single nucleotide polymorphism (SNP) and the primer or probe.

Disclosed are methods of treating venous thromboembolism (VTE) in a subject, the method comprising administering a venous thrombus size lowering therapy to the subject, wherein the venous thrombus size lowering therapy is a plasminogen activator inhibitor 1 (PAI-1) inhibitor wherein the subject has been diagnoses as being at risk of developing VTE by identifying the presence of a F5 Leiden pR506Q or a prothrombin G202010A single nucleotide polymorphism (SNP) present in a biological sample from the subject, said method comprising: obtaining a biological sample from the subject; and detecting whether a F5 Leiden pR506Q or a prothrombin G202010A single nucleotide polymorphism (SNP) is present in the sample by contacting the biological sample with primer or probe specific to a F5 Leiden pR506Q or a prothrombin G202010A single nucleotide polymorphism (SNP) and detecting binding between a F5 Leiden pR506Q or a prothrombin G202010A single nucleotide polymorphism (SNP) and the primer or probe, and diagnosing a subject as being at risk of developing VTE when a F5 Leiden pR506Q or a prothrombin G202010A single nucleotide polymorphism (SNP) is detected.

### C. Methods of Diagnosing and Treating

Disclosed are methods of diagnosing a subject as being at risk of developing VTE comprising identifying the presence of a F5 Leiden pR506Q or a prothrombin G202010A single nucleotide polymorphism (SNP) present in a biological sample from the subject. In some aspects, the method can further comprise administering a venous thrombus size lowering therapy to the subject, wherein the venous thrombus size lowering therapy is a plasminogen activator inhibitor (PAI-1) inhibitor. In some aspects, the method can further comprise administering a venous thrombus size lowering therapy to the subject diagnosing as being at risk of developing VTE, wherein the venous thrombus size lowering therapy is a plasminogen activator inhibitor (PAI-1) inhibitor.

Disclosed are methods of detecting of a F5 Leiden pR506Q or a prothrombin G202010A single nucleotide polymorphism (SNP) in a subject, said method comprising: obtaining a biological sample from the subject; and detecting whether a F5 Leiden pR506Q or a prothrombin G202010A single nucleotide polymorphism (SNP) is present in the biological sample by contacting the sample with primer or probe specific to a F5 Leiden pR506Q or a prothrombin G202010A single nucleotide polymorphism (SNP) and detecting binding between a F5 Leiden pR506Q or a prothrombin G202010A single nucleotide polymorphism (SNP) and the primer or probe. In some aspects, detecting a F5 Leiden pR506Q or a prothrombin G202010A SNP can comprise sequencing. In some aspects, detecting a F5 Leiden pR506Q or a prothrombin G202010A SNP can comprise a binding assay to determine if a probe binds to the SNP. In some aspects, the method can further comprise administering a venous thrombus size lowering therapy to the subject, wherein the venous thrombus size lowering therapy is a plasminogen activator inhibitor (PAI-1) inhibitor. In some aspects, the method can further comprise administering a venous thrombus size lowering therapy to the subject having a F5 Leiden pR506Q or a prothrombin G202010A single nucleotide polymorphism (SNP), wherein the venous thrombus size lowering therapy is a plasminogen activator inhibitor (PAI-1) inhibitor.

Disclosed are methods of diagnosing a subject as being at risk of developing VTE comprising identifying the presence of a F5 Leiden pR506Q or a prothrombin G202010A single nucleotide polymorphism (SNP) present in a biological sample from the subject, said method comprising: obtaining a biological sample from the subject; and detecting whether a F5 Leiden pR506Q or a prothrombin G202010A single nucleotide polymorphism (SNP) is present in the sample by contacting the biological sample with primer or probe specific to a F5 Leiden pR506Q or a prothrombin G202010A single nucleotide polymorphism (SNP) and detecting binding between a F5 Leiden pR506Q or a prothrombin G202010A single nucleotide polymorphism (SNP) and the primer or probe, and diagnosing a subject as being at risk of developing VTE when a F5 Leiden pR506Q or a prothrombin G202010A single nucleotide polymorphism (SNP) is detected. In some aspects, the method can further comprise administering a venous thrombus size lowering therapy to the subject, wherein the venous thrombus size lowering therapy is a plasminogen activator inhibitor (PAI-1) inhibitor.

Disclosed are methods of diagnosing and treating venous thromboembolism (VTE). Disclosed are methods of diagnosing and treating venous thromboembolism (VTE), the method comprising diagnosing the subject as being at greater risk of developing VTE; and administering a venous thrombus size lowering therapy to the subject, wherein the venous thrombus size lowering therapy is a plasminogen activator inhibitor (PAI-1) inhibitor.

In some aspects, diagnosing the subject as being at greater risk of developing VTE further comprises identifying whether a F5 Leiden pR506Q or a prothrombin G202010A single nucleotide polymorphism (SNP) are present in a biological sample from the subject.

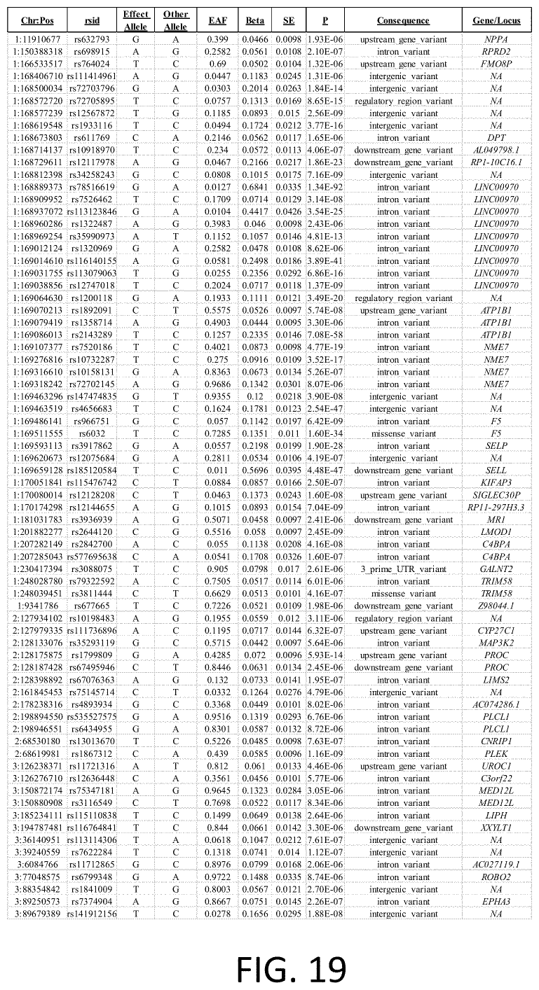

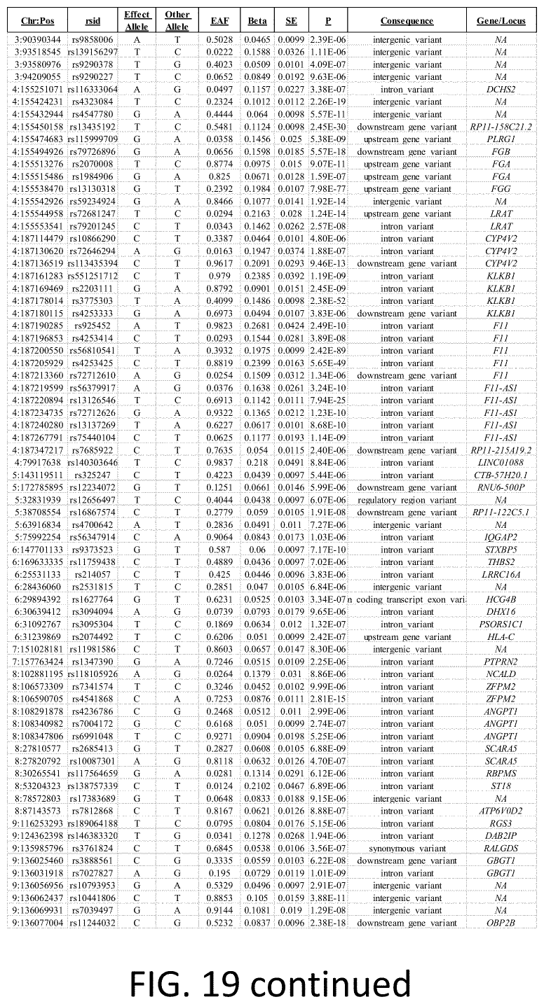

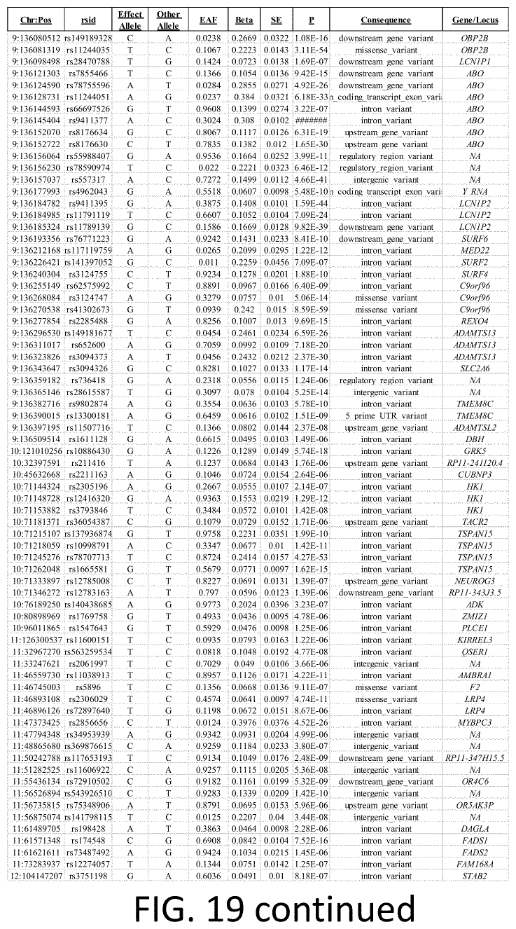

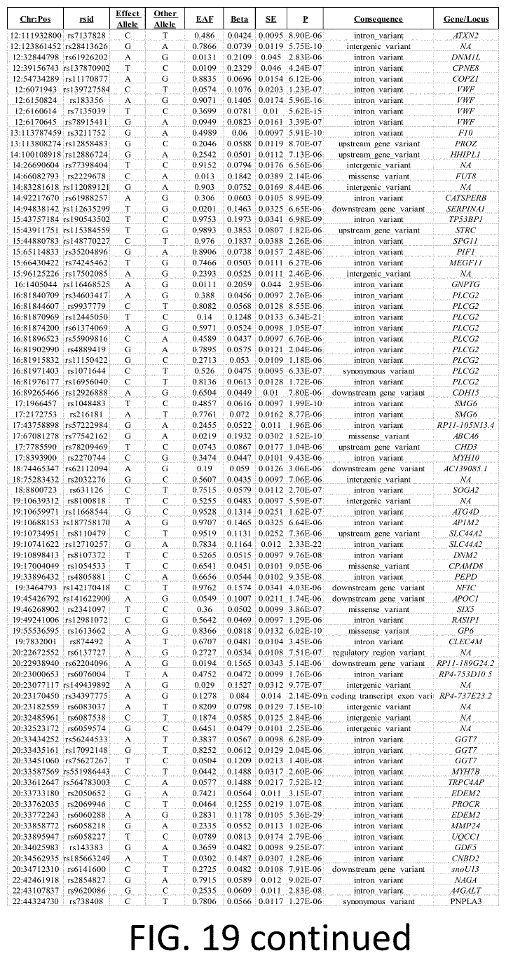

In some aspects, diagnosing the subject as being at greater risk of developing VTE further comprises identifying the presence of one or more of the 297 SNPs shown in FIG. 19 as present in a biological sample from the subject.

Disclosed are methods of diagnosing and treating comprising identifying whether a F5 Leidan pR506Q and a prothrombin G20210A single nucleotide polymorphism (SNP) are present in a biological sample from a subject; diagnosing the subject as being at risk for VTE if the F5 Leidan pR506Q and prothrombin G20210A mutations are present; and administering a venous thrombus size lowering therapy to the subject. In some aspects, the method can further comprise identifying the presence of one or more of the 297 SNPs identified in FIG. 19 are present in a biological sample from the subject.

Disclosed are methods of diagnosing and treating comprising identifying the presence of one or more of the 297 SNPs identified in FIG. 19 are present in a biological sample from the subject; diagnosing the subject as being at greater risk for VTE if one or more of the 297 SNPs identified in FIG. 19 are present; and administering a venous thrombus size lowering therapy to the subject.

In some aspects, the step of identifying or detecting any of the disclosed SNPs can be performed using techniques known in the art. For example, primers or probes that target the specific SNPs can be used. In some aspects, the probes can have a detectable label wherein the presence or absence of the detectable label confirms the presence or absence of a SNP. In some aspects, primers (labeled or not labeled) can be used to perform polymerase chain reaction (PCR) techniques. In some aspects, the primers and/or probes can be used in sequencing methods to identify the presence or absence of a SNP.

In some aspects, the venous thrombus size lowering therapy can comprise a PAI-1 inhibitor. In some aspects, a PAI-1 inhibitor can be, but is not limited to, vorapaxar tiplaxtinin (PAI-039), tiplatinin, TM5275, TVASSS, TVAVIS, TM5001, TM5007, TM5275, XR334, XR330, XR1853, XR5082, XR5118, XR11211, XR5967, AR-H029953XX, WAY-140312, HP129, XR1853, XR5118, diaplasinin (PAI-749), 535225, XK4044, T-1776Na, tannic acid, gallic acid, CDE-066, CDE-082, CDE-096, AZ3976, embelin, and bis-ANS. Additional PAI-1 inhibitors may be listed in A. Rouch et al., “Small molecules inhibitors of plasminogen activator inhibitor-1 An overview”, European Journal of Medicinal Chemistry 92 (2015) 619-636, which is incorporated by reference in its entirety.

In some aspects, the methods can further comprise administering a second therapy in addition to the venous thrombus size lowering therapy to the subject based on the diagnosis. In some aspects, the second therapy can be an LDL lowering therapy. In some aspects, the LDL lowering therapy can be a statin or a PCSK9 inhibitor. In some aspects, the second therapy can be an anticoagulant or a thrombolytic agent. In some aspects, an anticoagulant can be, but is not limited to, coumadin, dabigatran, rivaroxaban, apixaban. In some aspects, the second therapy can be a heparinoid therapy.

In some aspects, diagnosing the subject as being at greater risk of VTE further comprises calculating a polygenic risk score (PRS) based on the presence or absence of one or more of the 297 SNPs shown in FIG. 19, wherein the PRS is determined by summing a weighted risk value for each SNP.

In some aspects, the subject has been identified to have a PRS that corresponds to a high-risk group. In some aspects, a high PRS indicates a higher risk for developing VTE. For example, a PRS within the top 25, 20, 15, 10, 9, 8, 7, 6, 5, 4, 3, 2, 1, 0.5% of the population indicates the subject is at greater risk of developing VTE.

In some aspects, the biological sample can be any sample from the subject. For example, the biological sample can be, but is not limited to, blood, plasma, serum, urine, spinal fluid, sputum, cells, and tissue.

In some aspects, the subject can be a human. In some aspects, the subject can be a mammal.

### D. Methods of Determining

Disclosed are methods of detection and analysis of a large number of common genetic variants (e.g. SNPs) which can be used to calculate a polygenic risk score (PRS) suitable for identifying individuals at a greater risk of developing VTE.

Disclosed are methods of determining a PRS for developing VTE in a subject comprising identifying the presence of one or more of the 297 SNPs identified in FIG. 19 are present in a biological sample from the subject; and calculating the PRS by summing the weighted risk score associated with each SNP identified.

In some aspects, the methods further comprise identifying F5 Leidan pR506Q and prothrombin G20210A single nucleotide polymorphisms in the biological sample from the subject.

In some aspects, the step of identifying any of the disclosed SNPs can be performed using techniques known in the art. For example, primers or probes that target the specific SNPs can be used. In some aspects, the probes can have a detectable label wherein the presence or absence of the detectable label confirms the presence or absence of a SNP. In some aspects, primers (labeled or not labeled) can be used to perform polymerase chain reaction (PCR) techniques. In some aspects, the primers and/or probes can be used in sequencing methods to identify the presence or absence of a SNP.

In some aspects, a high PRS indicates a higher risk for developing VTE. For example, a PRS within the top 10, 9, 8, 7, 6, 5, 4, 3, 2, 1, 0.5% of the population indicates the subject is at greater risk of developing VTE.

In some aspects, the PRS is calculated by summing the weighted risk score associated with each SNP identified.

In some aspects, the disclosed methods can further comprise diagnosing the subject as at risk of developing VTE based on the PRS, and administering a venous thrombus size lowering therapy to the subject based on the diagnosis.

In some aspects, the biological sample can be any sample from the subject. For example, the biological sample can be, but is not limited to, blood, plasma, serum, urine, spinal fluid, sputum, cells, and tissue.

In some aspects, the subject can be a human. In some aspects, the subject can be a mammal.

In some aspects, the PRS determined in accordance with the present disclosure can also assist in providing an indication of how likely it is that a patient will respond to any particular therapy for the treatment of VTE, particularly PAI-1 inhibitors. In some aspects, disclosed are methods of the identification of a patient population for testing treatment options for preventing or slowing down the development of VTE based on the PRS disclosed herein.

In some aspects, the presence of a high genetic propensity to VTE can be treated as a warning to commence prophylactic or therapeutic treatment. For example, individuals with elevated risk of developing VTE can be monitored differently (e.g., more frequently) or can be treated prophylactically (e.g., with one or more drugs or surgery). Presence of a high propensity to develop VTE can also indicate the utility of performing secondary testing, such as CT scan and other methods known in the art.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for diagnosing, detecting or treating VTE, the kit comprising primers, probes, or antibodies that bind to a F5 Leidan pR506Q or a prothrombin G20210A SNP or one or more of the 297 SNPs identified in FIG. 19.

### Examples

1. Results

Venous thromboembolism (VTE) is a complex disease impacted by both environmental1 and genetic determinants, and the narrow-sense heritability of VTE has been estimated to be approximately 30%. At the time of analysis, genome-wide association studies (GWAS) revealed only 11 loci reaching genome-wide significance, leaving a significant portion of VTE heritability unknown.

Large-scale biobanks linking genetic and diverse phenotypic data in the electronic health record (EHR) are being developed throughout the world. Leveraging two large-scale biobanks—UK Biobank and the Million Veteran Program (MVP)—this study aimed to: 1) perform a genetic discovery analysis for VTE, 2) evaluate the causal role of blood lipids in VTE, 3) further characterize the role of plasminogen activator inhibitor-1 (PAI-1) in VTE, and 4) develop and evaluate a genome-wide polygenic risk score (PRS) for VTE.

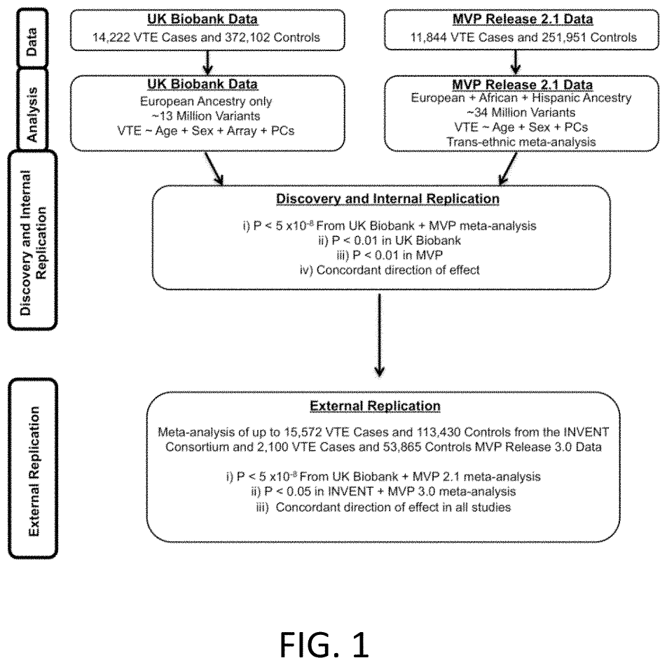

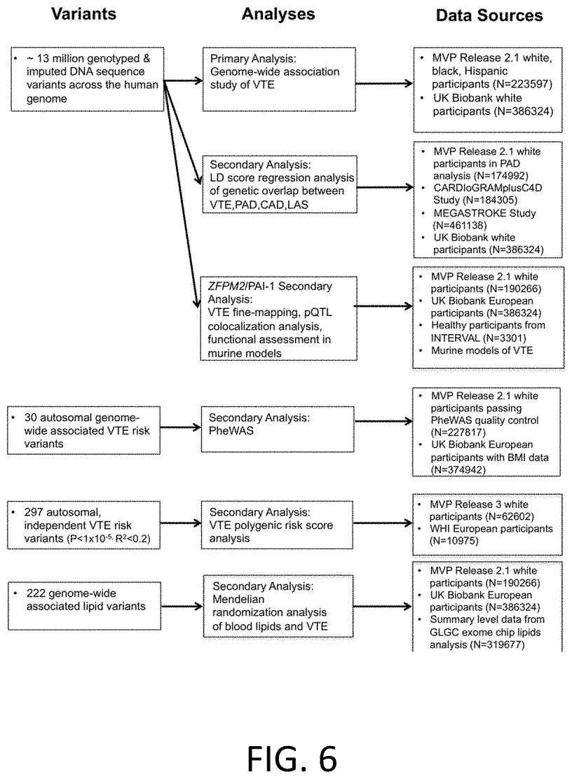

A two-phased VTE discovery GWAS was designed (FIG. 1, FIG. 6). FIG. 1 shows an association analysis was performed for DNA sequence variants in 14,222 VTE cases and 372,102 controls of European ancestry using logistic regression. These results were combined with association statistics from DNA sequence variants across 3 mutually exclusive ancestry groups in the Million Veteran Program release 2.1 data representing 11,844 VTE cases and 251,951 controls. Data from UK Biobank and MVP were meta-analyzed using an inverse-variance weighted fixed effects method. A significance threshold of two-sided P<5×10⁻⁸ (genome-wide significance) was set, and also an internal replication two-sided P<0.01 was required in each of the MVP and UK Biobank analyses, with concordant direction of effect, to minimize false positive findings. Subsequently, external replication was performed using summary data from the INVENT consortium (up to 15,572 VTE cases and 113,430 controls) meta-analyzed with data from MVP 3.0 (2,100 VTE cases and 53,865 controls), requiring an external replication P<0.05 with a consistent direction of effect. FIG. 6 shows the primary analysis consisted of a genome-wide association study to identify novel VTE risk variants. Secondary analyses included: an analysis of VTE and atherosclerosis overlap, a fine-mapping analysis, colocalization analysis, and functional analysis of PAI-1 using trans-ethnic summary statistics, pQTL data, and murine models respectively, a closer examination of autosomal VTE risk variants through PheWAS, generation and analysis of a 297 variant VTE polygenic risk score, and a Mendelian randomization analysis of blood lipids and VTE. In Phase 1, MVP release 2.1 data was used and testing for association separately among individuals of European (whites), African (blacks), and Hispanic ancestry was performed and results across ancestral groups were meta-analyzed. In UK Biobank, association testing was performed in individuals of European ancestry. Statistical evidence across MVP and UK Biobank was combined and a significance threshold of P<5×10⁻⁸ (genome-wide significance) was set, and an internal replication P<0.01 also required in each of the individual MVP and UK Biobank analyses, with concordant directions of effect, to minimize false positive findings. In Phase 2, an additional round of external replication was performed for lead variants using summary data of up to 15,572 VTE cases and 113,430 disease-free controls from the INVENT consortium combined with 2,100 VTE cases and 53,865 controls from MVP 3.0 data, requiring P<0.05 with consistent direction of effect for successful replication.

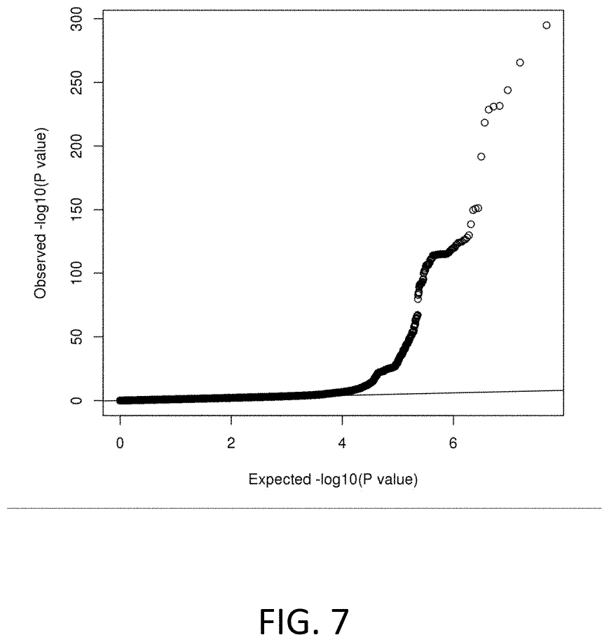

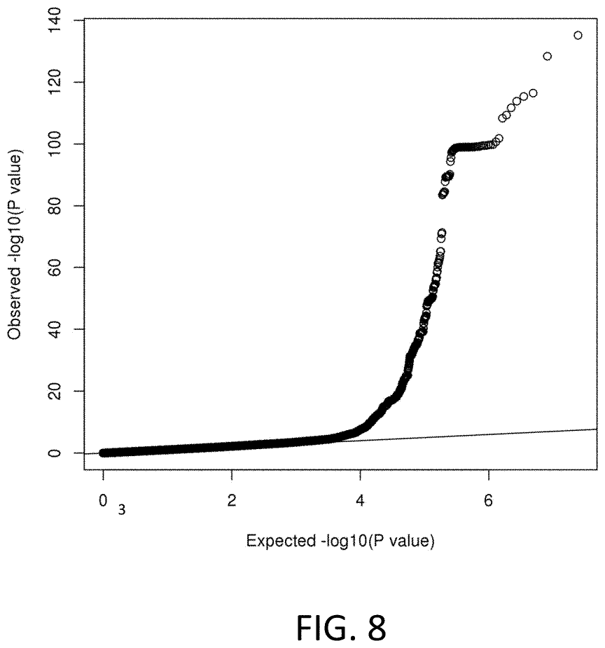

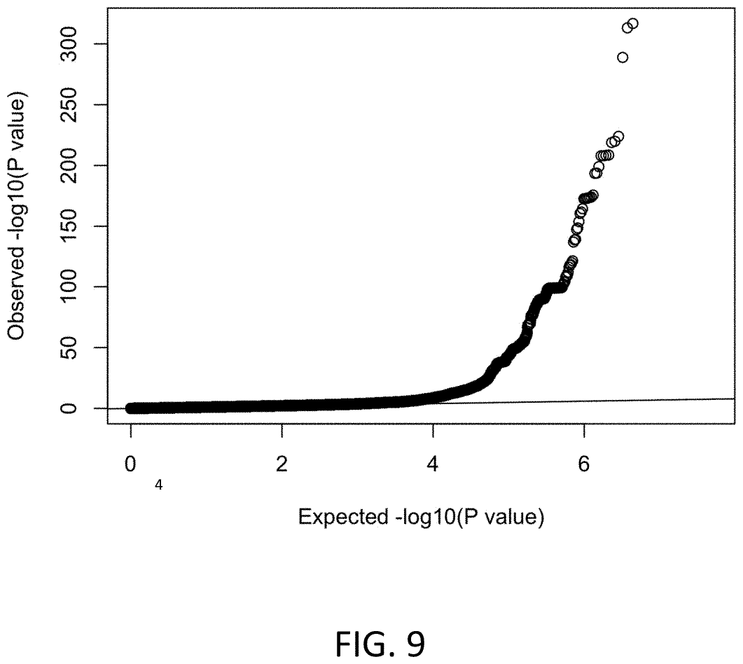

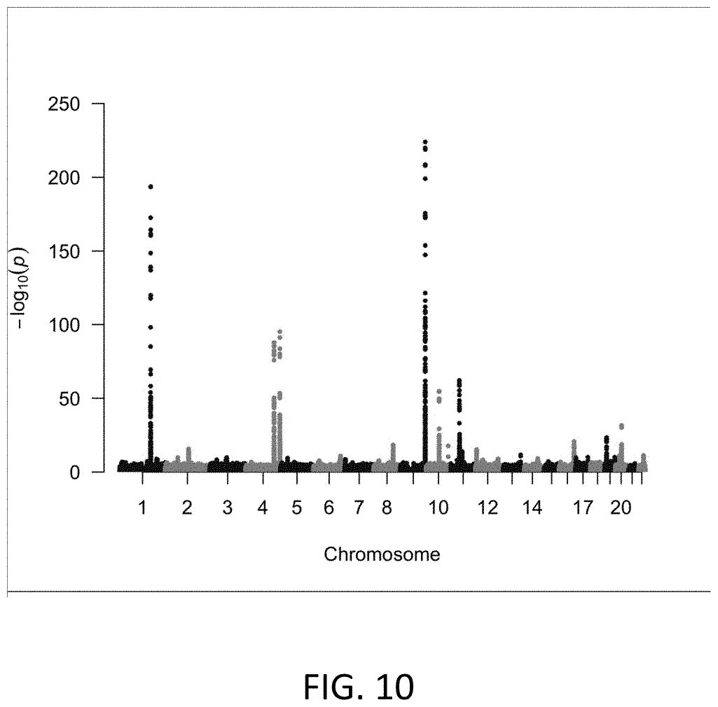

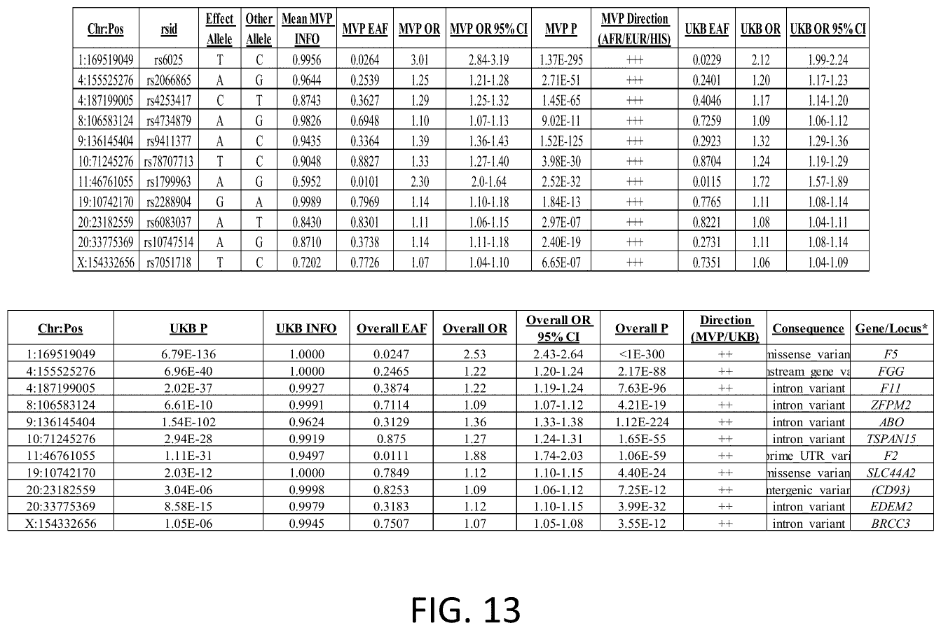

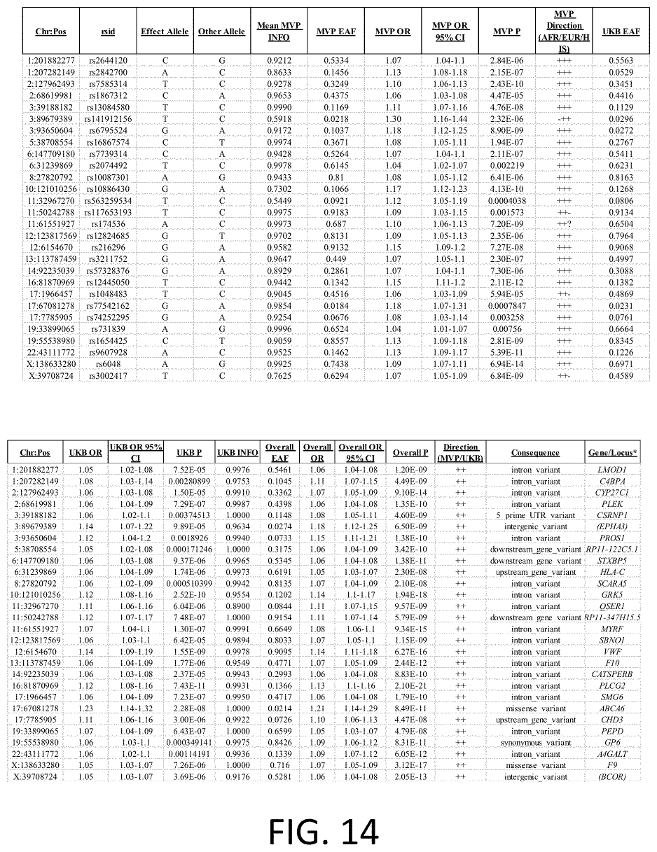

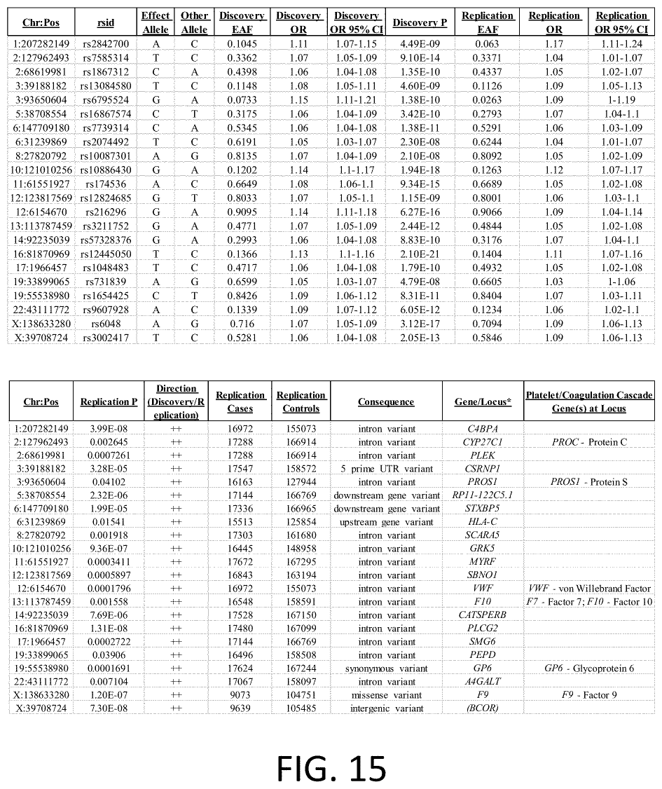

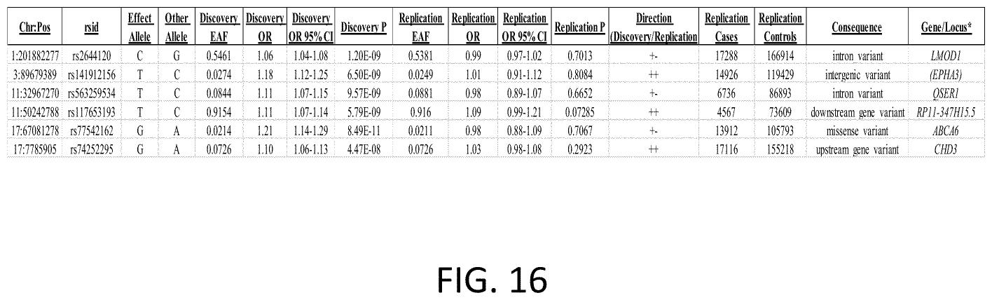

In MVP, the discovery analysis was composed of 11,844 VTE cases (8,929 white, 2,261 black, 654 Hispanic) and 211,753 controls from the MVP release 2.1 data. In UK Biobank 14,222 VTE cases and 372,102 controls were identified. The baseline characteristics for both cohorts are presented in Tables 1-2. VTE cases were more likely to be older, have a history of smoking, a higher body-mass index, and have type 2 diabetes. Following trans-ethnic meta-analysis across MVP and UK Biobank, a total of 2,706 variants at 39 loci met a genome-wide significance threshold, with P<0.01 and concordant effect directions in both datasets (FIG. 7-10). Quantile-quantile plots were inspected for ancestry-specific analyses in MVP (European/African/Hispanic) and genomic control values were <1.05 for each racial group. In FIG. 7, no systemic inflation was observed (λ_{Gc}=1.04). All P values were two-sided. In FIG. 8, No systemic inflation was observed (λ_{GC}=1.08). In FIG. 9, the expected logistic regression association P values versus the observed distribution of P values for VTE association (Wald statistic) are displayed. No systemic inflation was observed (λ_{GC}=1.06). All P values were two-sided. In a linkage disequlibrium (LD) score regression analysis restricted to Europeans (N=23,151 VTE cases and 553,439 controls), the LD score intercept was observed to equal 1.02, indicating nearly all the inflation in test statistics is due to genuine polygenicity in VTE as a trait. The F5 Leiden variant, rs6025 (p.R506Q, NC_000001.10:g.169519049T>C), was the top association result (2.5% frequency for the T allele; OR=2.53; 95% CI: 2.43-2.64; P<1.0×10⁻³⁰⁰). All 11 previously described genome-wide VTE loci were replicated, and 28 candidate novel VTE loci brought forward for external replication were identified (FIG. 13 and FIG. 14). Of the 28 candidate novel loci, 22 successfully replicated in an independent set of up to 17,672 VTE cases and 167,295 controls (FIG. 15 and FIG. 16).

TABLE 1
Demographic and clinical characteristics for individuals
in the UK Biobank VTE GWAS analysis

| | VTE Cases | VTE Controls |
|---|---|---|
| N Individuals | 14,222 | 372,102 |
| Age ± SD, years | 60.3 ± 7.1 | 57.3 ± 7.9 |
| Male, n (%) | 6,374 (44.8%) | 172,438 (46.3%) |
| Former Smoker, n (%) | 5,517 (38.8%) | 130,965 (35.2%) |
| Current Smoker, n (%) | 1,767 (12.4%) | 37,878 (10.2%) |
| Hypertension, n (%) | 6,441 (45.3%) | 119,426 (32.1%) |
| Diabetes, n (%) | 1,436 (10.1%) | 16,519 (4.4%) |
| Hyperlipidemia, n (%) | 3,778 (26.6%) | 63,964 (17.2%) |
| Body-Mass Index ± SD, kg/m² | 29.0 ± 5.5 | 27.3 ± 4.7 |

| | |
|---|---|
| Variants Included in Analysis | 13,599,453 |

TABLE 2
Demographic and clinical characteristics for veterans in the MVP VTE GWAS analysis

| | White | | Black | | Hispanic | |
|---|---|---|---|---|---|---|
| | VTE | VTE | VTE | VTE | VTE | VTE |
| | Cases | Controls | Cases | Controls | Cases | Controls |
| N Veterans | 8,929 | 181,337 | 2,261 | 49,400 | 654 | 21,214 |
| Age ± SD, years | 71.0 ± 11.3 | 68.0 ± 12.8 | 66.1 ± 11.5 | 61.5 ± 11.6 | 66.8 ± 13.0 | 60.5 ± 14.7 |
| Male, n (%) | 8,490 | 168,161 | 2,402 | 42,722 | 613 | 19,258 |
| | (95.0%) | (92.7%) | (91.6%) | (86.5%) | (93.7%) | (90.8%) |
| Current Smoker, | 1,697 | 32,814 | 680 | 13,505 | 77 | 3,399 |
| n (%) | (19.0%) | (18.1%) | (25.9%) | (27.3%) | (11.8%) | (16.0%) |
| Former Smoker, | 5,122 | 98,706 | 1,244 | 21,217 | 398 | 10,317 |
| n (%) | (57.4%) | (54.4%) | (47.5%) | (42.9%) | (60.9%) | (48.6%) |
| Diabetes, n (%) | 3,742 | 62,283 | 1,387 | 20,857 | 342 | 8,431 |
| | (41.9%) | (34.3%) | (52.9%) | (42.2%) | (52.3%) | (39.7%) |
| Hyperlipidemia, | 3,812 | 80,417 | 1,057 | 19,515 | 291 | 8,118 |
| n (%) | (42.7%) | (44.3%) | (40.3%) | (39.5%) | (44.5%) | (38.3%) |
| Body-Mass Index ± | 31.5 ± 6.7 | 30.3 ± 5.9 | 31.3 ± 7.0 | 30.5 ± 6.2 | 32.2 ± 7.0 | 30.8 ± 5.8 |
| SD, kg/m² | | | | | | |

| | | | |
|---|---|---|---|
| Variants Included | 19,972,400 | 31,960,759 | 28,192,968 |
| in Analysis | | | |

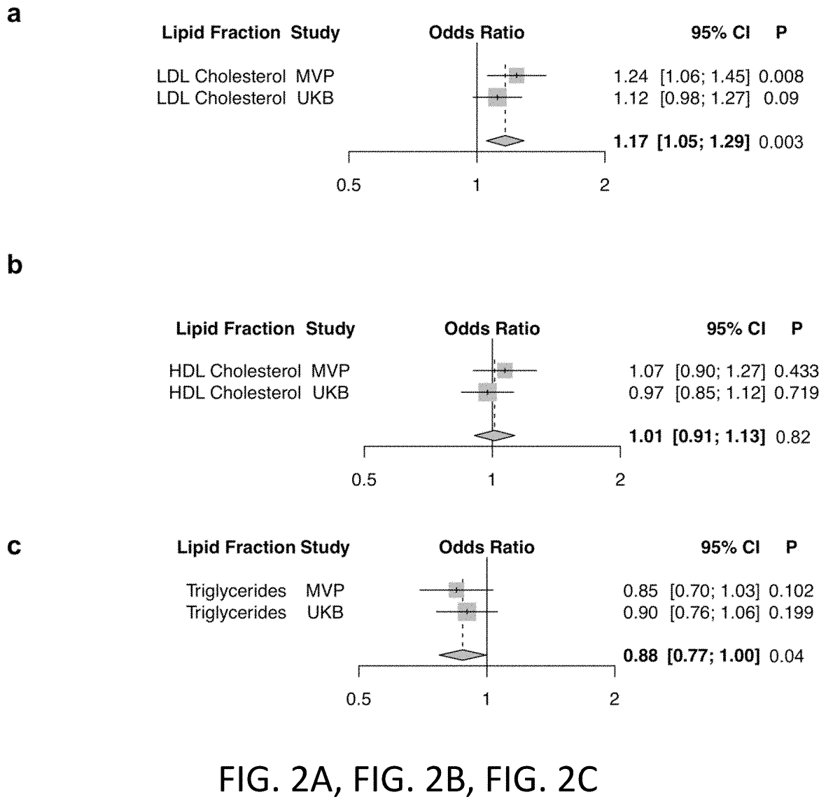

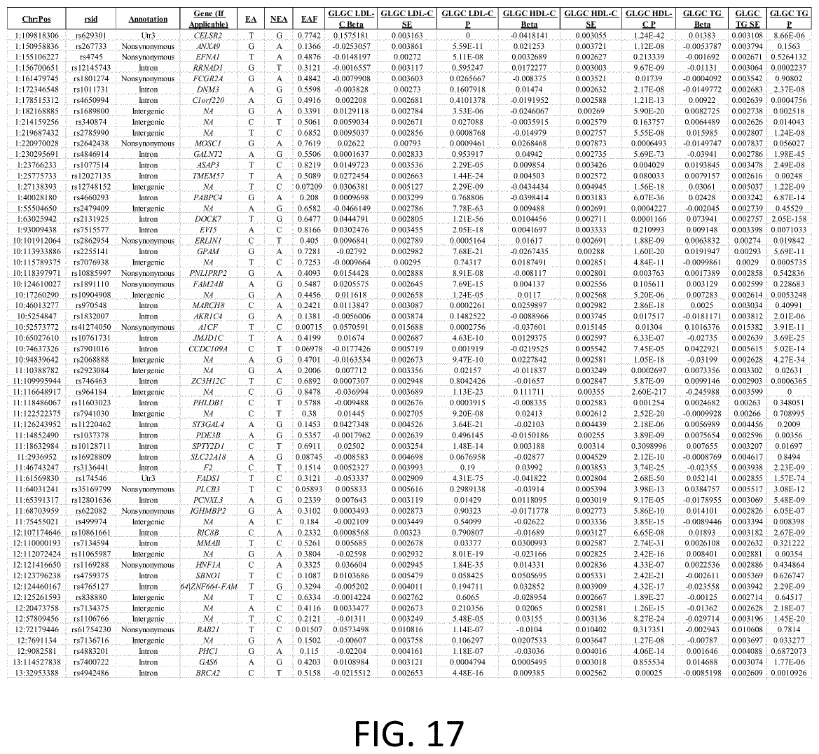

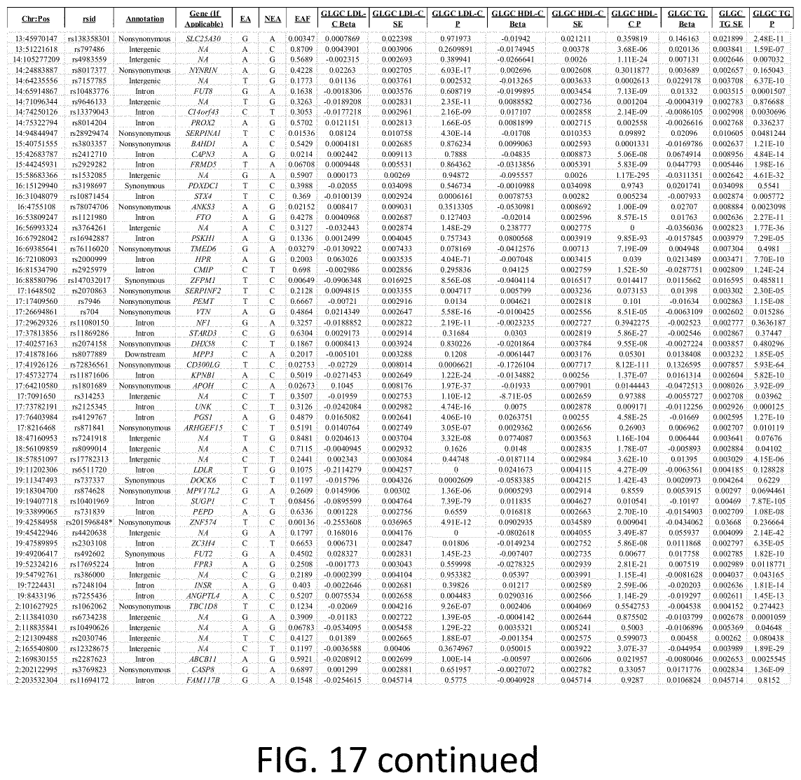

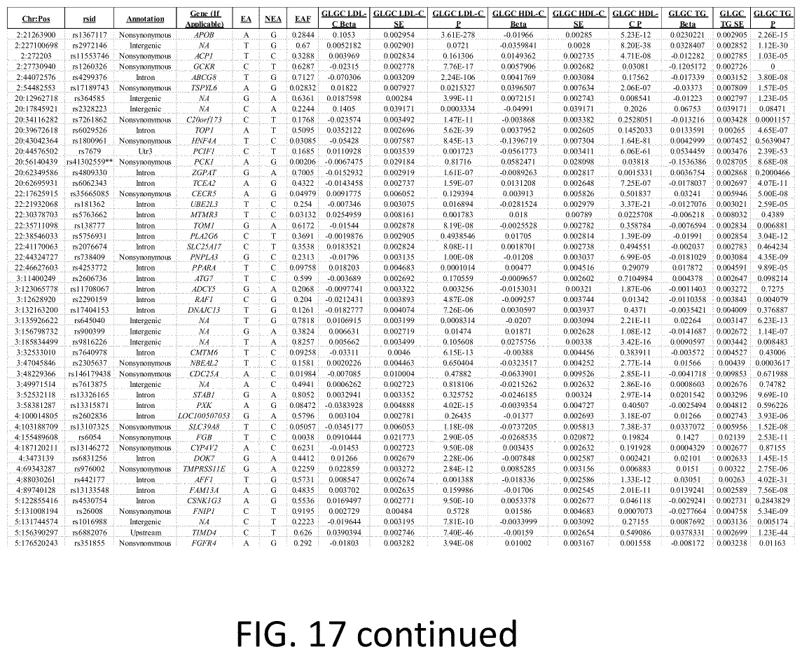

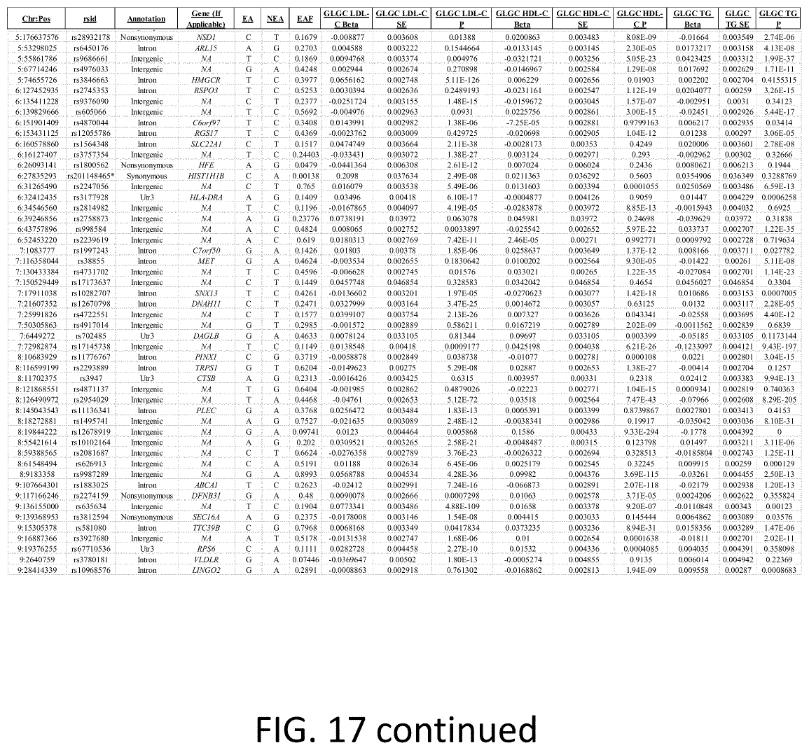

One large randomized controlled trial showed that LDL cholesterol-lowering with a statin versus placebo led to a reduced risk of venous thromboembolic events. Causal relationships of blood lipids with VTE development were investigated by performing a multivariate Mendelian randomization analysis using a weighted polygenic score of 222 lipid-associated variants from the Global Lipids Genetics Consortium and summary data from the MVP release 2.1 and UK Biobank VTE GWAS restricted to Europeans (FIG. 17). A 1-standard deviation of genetically-elevated LDL cholesterol was associated with an increased risk of VTE (ORLDL=1.17, 95% CI=1.05-1.29, PLDL=0.003). In contrast, both a 1-standard deviation of genetically-elevated HDL cholesterol and a 1-standard deviation of genetically-elevated triglycerides were not associated with risk of VTE [ORHDL=1.01, 95% CI=0.91-1.13, PHDL=0.82; ORTriglycerides=0.88, 95% CI=0.77-1.00, PTriglycerides=0.04] after Bonferroni correction (P<0.016=[0.05/3 lipid fractions]). An MR-Egger analysis indicated no pleiotropic biases of the lipid genetic instruments [MR-Egger intercept P>0.05 for all 3 lipid fractions (Table 3, FIG. 2)].

TABLE 3
Logistic regression (Wald statistic) association effect estimates, standard and errors, and
two-sided P values for the 222 variant lipid genetic risk score Mendelian randomization
analysis with venous thromboembolism (VTE) risk. The 4 different Mendelian randomization
(MR) methods used to determine this association were conventional inverse weighted MR,
MR-Egger, weighted median MR, and multivariable MR. Summary-level lipids data from up
to 319,677 participants of the Global Lipids Genetics Consortium21, and VTE association
data from MVP (N = 8,929 cases; 181,337 controls) and UK Biobank (N = 14,222 cases;
372,102 controls) were used for this analysis.

| Lipid Fraction | MR Method | Study | Beta | SE | P |
|---|---|---|---|---|---|
| LDL Cholesterol | Inverse-variance | MVP | 0.167 | 0.079 | 0.034 |
| | weighted | | | | |
| LDL Cholesterol | Multivariable | MVP | 0.215 | 0.081 | 0.008 |
| LDL Cholesterol | MR-Egger | MVP | 0.22 | 0.098 | 0.025 |
| LDL Cholesterol | MR-Egger Intercept | MVP | | | 0.36 |
| HDL Cholesterol | Inverse-variance | MVP | 0.124 | 0.075 | 0.095 |
| | weighted | | | | |
| HDL Cholesterol | Multivariable | MVP | 0.068 | 0.087 | 0.433 |
| HDL Cholesterol | MR-Egger | MVP | 0.11 | 0.094 | 0.22 |
| HDL Cholesterol | MR-Egger Intercept | MVP | | | 0.88 |
| Triglycerides | Inverse-variance | MVP | −0.144 | 0.084 | 0.088 |
| | weighted | | | | |
| Triglycerides | Multivariable | MVP | −0.165 | 0.101 | 0.102 |
| Triglycerides | MR-Egger | MVP | −0.2 | 0.11 | 0.058 |
| Triglycerides | MR-Egger Intercept | MVP | | | 0.375 |
| LDL Cholesterol | Inverse-variance | UK | 0.086 | 0.064 | 0.181 |
| | weighted | Biobank | | | |
| LDL Cholesterol | Multivariable | UK | 0.11 | 0.067 | 0.09 |
| | | Biobank | | | |
| LDL Cholesterol | MR-Egger | UK | 0.068 | 0.081 | 0.398 |
| | | Biobank | | | |
| LDL Cholesterol | MR-Egger Intercept | UK | | | 0.71 |
| | | Biobank | | | |
| HDL Cholesterol | Inverse-variance | UK | 0.014 | 0.061 | 0.82 |
| | weighted | Biobank | | | |
| HDL Cholesterol | Multivariable | UK | −0.026 | 0.072 | 0.72 |
| | | Biobank | | | |
| HDL Cholesterol | MR-Egger | UK | 0.02 | 0.078 | 0.798 |
| | | Biobank | | | |
| HDL Cholesterol | MR-Egger Intercept | UK | | | 0.481 |
| | | Biobank | | | |
| Triglycerides | Inverse-variance | UK | −0.061 | 0.07 | 0.382 |
| | weighted | Biobank | | | |
| Triglycerides | Multivariable | UK | −0.108 | 0.084 | 0.19 |
| | | Biobank | | | |
| Triglycerides | MR-Egger | UK | −0.079 | 0.088 | 0.365 |
| | | Biobank | | | |
| Triglycerides MR-Egger Intercept | | UK | | | 0.73 |
| | | Biobank | | | |

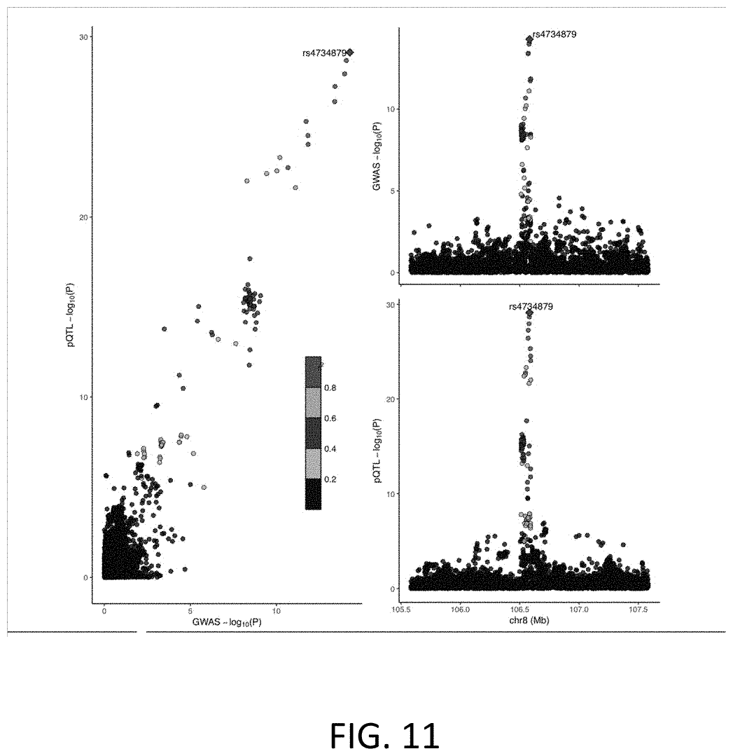

Given the known role of PAI-1 in venous thrombosis and fibrinolysis in model systems, the ZFPM2 VTE GWAS and the PAI-1 trans-pQTL associations were thought to represent colocalizing signals at the ZFPM2 locus. A colocalization analysis pipeline was used to compute the colocalization posterior probability (CLPP) for the ZFPM2 locus. Using European MVP release 2.1 and UK Biobank European VTE meta-analyzed summary statistics, PAI-1 pQTL results in human plasma from the INTERVAL study, and reference LD information of 503 European participants from 1000 Genomes phase 3 whole genome sequencing data, a CLPP of 0.203 was calculated at this locus. Previous work suggests that a CLPP>0.01 is indicative of a “reasonably high” probability of colocalization, and the LocusCompare plot at this site further indicates that the ZFPM2 VTE GWAS and PAI-1 pQTL associations likely represent a true colocalization event (FIG. 11). The pQTL p-values were derived from the plasma samples of 3,301 participants of the INTERVAL¹⁹ study based on a linear regression model. The GWAS p-values were derived from a logistic regression model (Wald statistic) and meta-analysis from the current study.

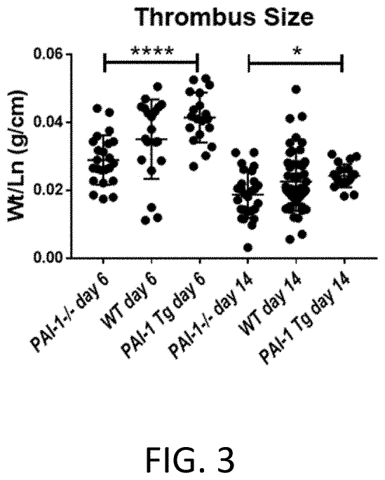

PAI-1 influences thrombosis by directly inhibiting conversion of plasminogen to plasmin and indirectly via disrupting the interaction of circulating monocytes with glycoprotein vitronectin within the thrombus and adjacent vein wall. Monocytes are a key source of factor III (tissue factor) as well as matrix metalloproteinases during thrombus clearance. Given the colocalization between PAI-1 concentration and human VTE, this study sought experimentally to determine the impact of PAI-1 levels on venous thrombus size in an experimental DVT model utilizing transgenic mice. PAI-1−/− mice have no circulating active PAI-1, whereas those overexpressing PAI-1 (PAI-1 Tg), have levels approximately 137-fold greater than wild-type C57B/L6 (WT) mice. At 6 days following IVC occlusion with generation of thrombus, the PAI-1 overexpressing mice had 1.5-fold larger thrombus size compared to PAI-1−/− mice, with the WT mice demonstrating an intermediate phenotype. This difference persists during late thrombus resolution, at day 14 (FIG. 3), demonstrating progressive impairment in thrombus clearance in the setting of increasing PAI-1 protein levels. Inferior vena cava venous thrombus size was measured at day 6 and day 14 after inferior vena cava ligation in PAI-1 Tg (day 6 N=19; day 14 N=20), wild type (day 6 N=20; day 14 N=49), and PAI-1 −/− mice (day 6 N=23; day 14 N=27). Thrombus size was observed to be larger in the PAI-1 Tg mice compared to PAI-1−/− mice (one-way analysis of variance followed by Tukey's multiple comparisons post hoc test, *p=0.02, ****p<0.0001). A scatter dot plot depicting mean thrombus size±standard deviation is shown.

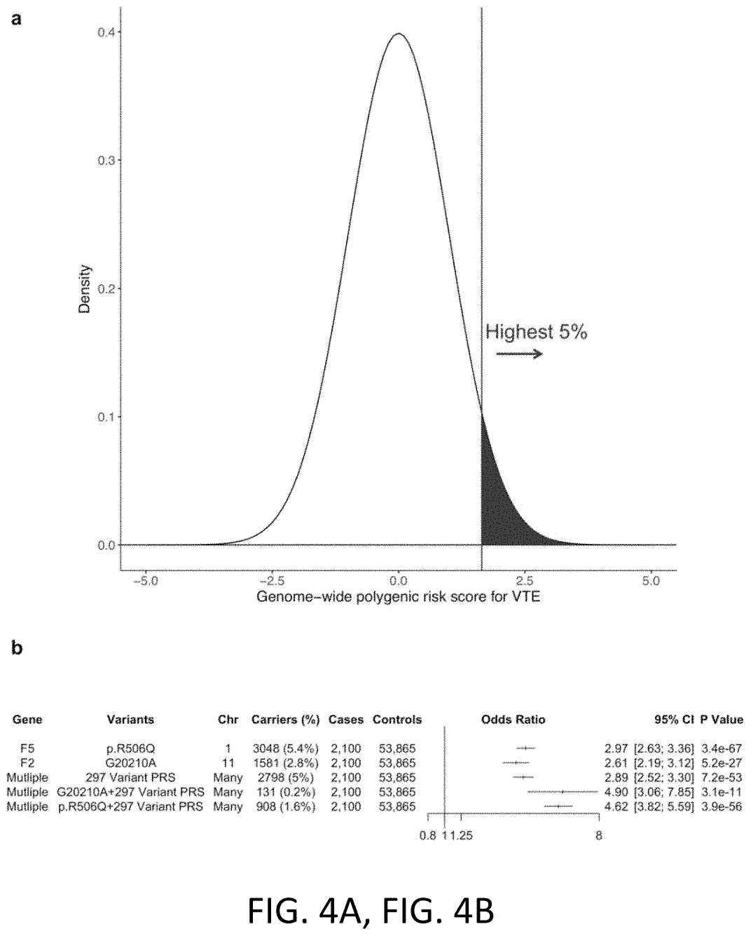

Finally, the contribution of polygenic inheritance on VTE risk were examined. Currently, the F5 Leiden (p.R506Q) and F2 (prothrombin) G20210A mutations, low-frequency variants which confer a 2-3-fold risk of VTE, are frequently tested in clinical settings to evaluate the role of inherited thrombophilia predisposing to acute thrombotic syndromes. Given the individual associations of common genetic variants with VTE, heritable VTE risk may also be explained by an aggregate of common variant VTE nsk alleles. Those at the right tail of the normally distributed VTE PRS (highest 5%) would be at significantly increased VTE risk (FIG. 4a).

A 297-variant VTE PRS was generated using a pruning and thresholding method (R2<0.2, P<1×10⁻⁵) from European MVP release 2.1 and UK Biobank European VTE meta-analyzed summary statistics (FIG. 19). Notably, the LD blocks (R2>0.2) containing the F5 p.R506Q and F2 G20210A variants were excluded from the PRS. The associated VTE risk was assessed for the 5% of individuals with the highest PRSVTE relative to the rest of the population using prevalent data from MVP release 3.0, a set of 2,100 VTE cases and 53,865 VTE controls entirely independent from the individuals in the MVP discovery GWAS. It was observed that the 2,798 individuals in MVP release 3.0 with the 5% highest PRSVTE had 2.89-fold increased risk of VTE relative to the rest of the population (ORPRS=2.89, 95% CI=2.52-3.30, PPRS=7.2×10⁻³). This effect estimate was similar in magnitude to those observed for F5 p.R506Q (ORFS=2.97, 95% CI=2.63-3.36, PF5=3.4×10-67) and F2 G20210A (ORF2=2.61, 95% CI=2.19-3.12, PF2=5.2×10-27) [FIG. 4b]. In addition, it was observed that this risk was further compounded for individuals among the top 5% with increased polygenic VTE risk who were also F5 Leiden or F2 G20210A carriers.

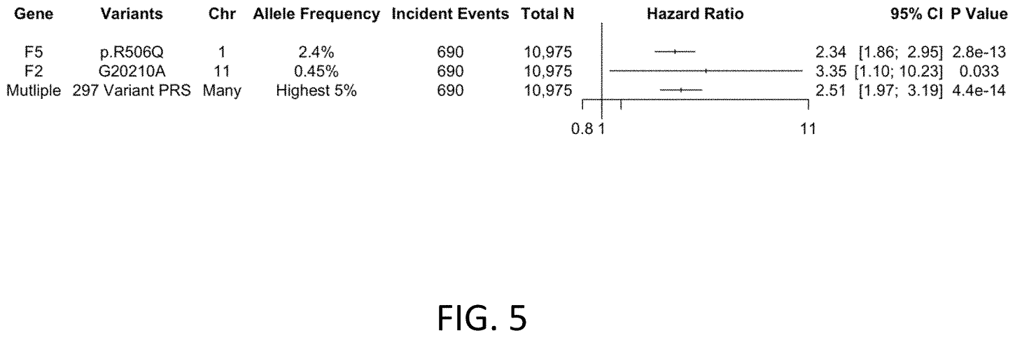

Replication of the PRS findings using incident VTE data from the prospective Women's Health Initiative (WHI) Hormone Trial (HT) was investigated. In total, among 10,975 European women prospectively followed for up to 25 years in the WHI-HT, 690 incident VTE events were identified among participants with genetic data. Demographic and clinical characteristics for WHI participants in our VTE incident event analysis are shown in Table 4. The risk for carriers of F5 p.R506Q and F2 G20210A mutations as well as those among the 5% highest PRSVTE was estimated through Cox proportional hazards models. It was observed that F5 p.R506Q carriers were at greater than 2-fold risk of developing VTE [Hazard Ratio (HRFS)=2.34, 95% CI=1.86-3.35, PF5=2.8×10⁻¹³], and the F2 G20210A mutation was nominally associated with increased VTE risk [HRF2=3.35, 95% CI=1.10-10.23, PF2=0.033]. The 549 individuals in WHI with the 5% highest PRSVTE had 2.51-fold risk of incident VTE relative to the rest of the population [HRPRS=2.51, 95% CI=1.97-3.19, PPRS=4.4×10⁻¹⁴] as depicted in FIG. 5. Much like in MVP, the risk among the 5% of the population with the highest PRSVTE in WHI was comparable in effect size to that of large-effect, monogenic mutations in F5 and F2.

TABLE 4
Demographic and clinical characteristics of Women's Health Initiative
study participants stratified by sub-study.

| | Women's Health Initiative Sub-Study | | |
|---|---|---|---|
| | GARNET | LLS | MS |
| N | 4,233 | 1,105 | 5,637 |
| Incident VTE Cases, | 457 (10.8%) | 53 (4.8%) | 180 (3.2%) |
| n (%) | | | |
| Age ± SD, years | 65 ± 6.9 | 68 ± 3.4 | 68 ± 5.9 |
| Body Mass Index ± SD, | 29.8 ± 6.8 | 27.7 ± 4.8 | 28.4 ± 6.5 |
| kg/m² | | | |
| Current Smoker, n (%) | 462 (10.9%) | 46 (4.2%) | 402 (7.1%) |
| Former Smoker, n (%) | 1,610 (38.0%) | 431 (39.0%) | 2,289 (40.6%) |
| Diabetes, n (%) | 134 (3.2%) | 32 (2.9%) | 341 (6.0%) |
| Statin Use, n (%) | 369 (8.7%) | 73 (6.6%) | 548 (9.7%) |
| Current use of Hormone | 333 (7.9%) | 95 (8.6%) | 366 (6.5%) |
| Therapy, n (%) | | | |
| Former use of Hormone | 1,214 (28.7%) | 290 (26.2%) | 1,423 (25.2%) |
| Therapy, n (%) | | | |
| Hyperlipidemia, n (%) | 3,231 (76.3%) | 884 (80.0%) | 4,260 (75.6%) |

Abbreviations: VTE, Venous Thromboembolism; SD, Standard Deviation; GARNET, Genomics and Randomized Trials Network; LLS, Long Life
Study; MS, Memory Study

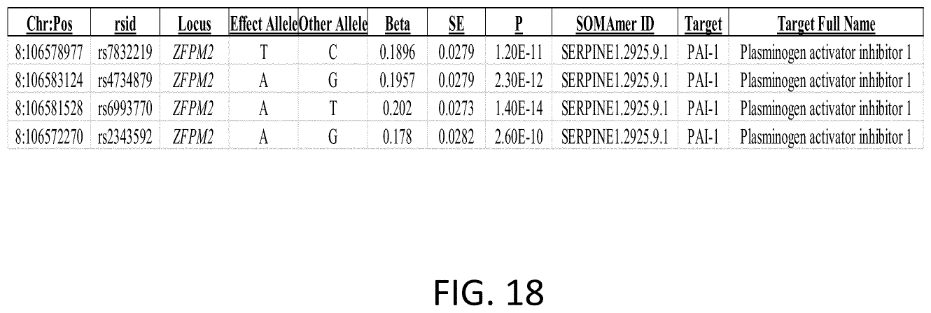

A PheWAS (1), an analysis of how DNA sequence variants differ in their contribution to vascular disease risk in the arterial and venous territories (2), an examination of VTE risk variant-pQTL associations (3), and results of a VTE fine-mapping analysis including a 99% credible set of 4 variants at the ZFPM2 locus which were genome-wide trans-pQTL associations with plasma PAI-1 concentration (FIG. 18) are discussed herein.

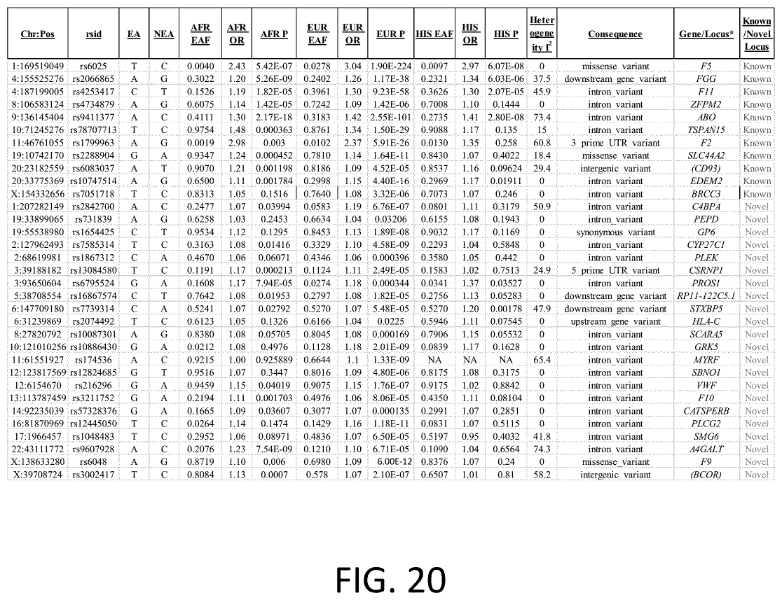

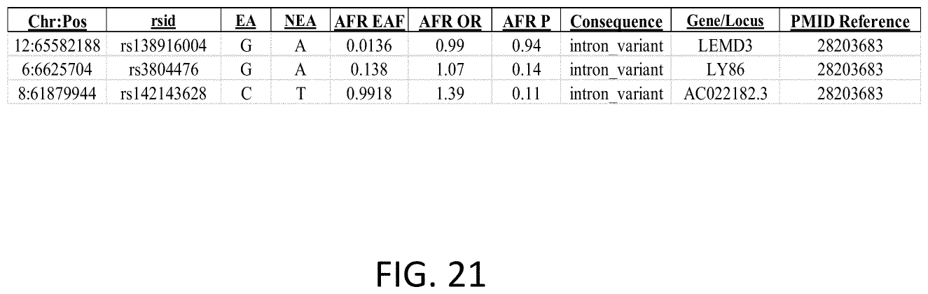

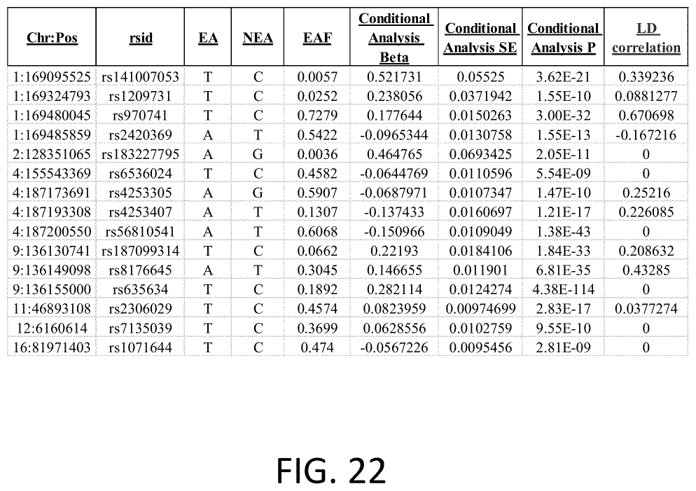

Of the 22 novel loci, 6 contained at least one gene implicated in the coagulation cascade or platelet function (FIG. 15). Three previously reported suggestive (5.0×10−8<P<0.05) VTE associations at the GP64, STXBP55, and VWFS loci were now observed at genome-wide significance. Across all 33 VTE loci (11 known and 22 novel), 31 were directionally consistent across whites, blacks, and Hispanics in MVP and 22 demonstrated at least nominal significance (P<0.05) in blacks and 7 in Hispanics (FIG. 20). 2 known and 4 novel VTE loci demonstrated moderate heterogeneity across the three ethnicities (50%<heterogeneity 12<75%), but remained below the pre-specified heterogeneity threshold of 75%. In addition, no evidence was found of association for 3 African specific variants previously reported in an analysis of 393 African ancestry VTE cases that lacked independent replication (FIG. 21). In a conditional analysis using combined summary statistics from MVP Europeans and UK Biobank, an additional 15 independent VTE variants were identified across the 33 loci (FIG. 22).

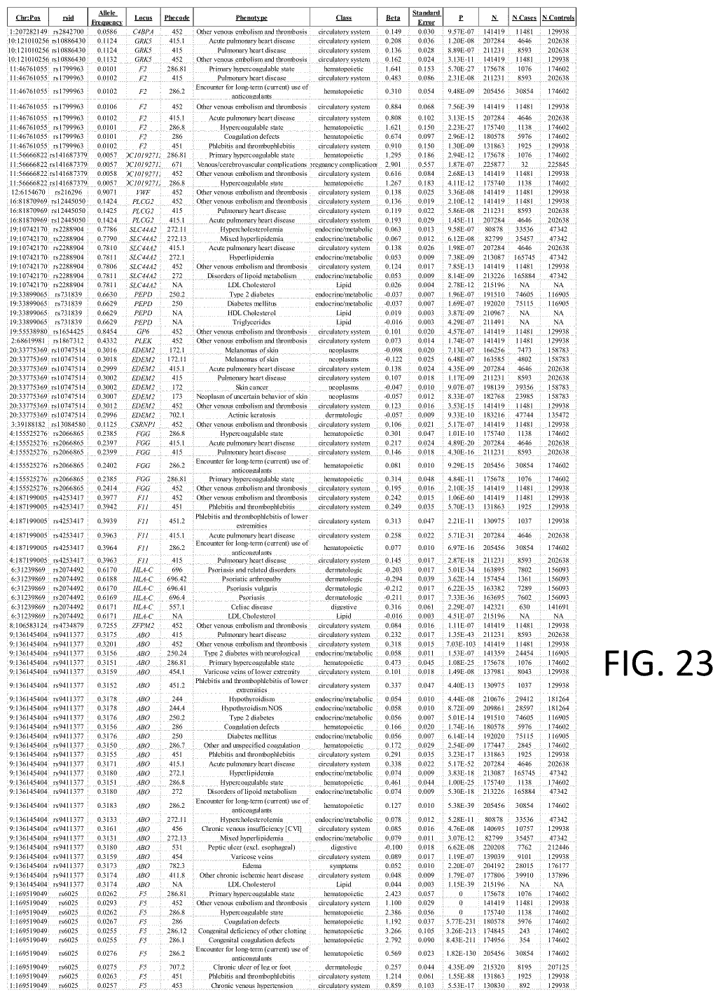

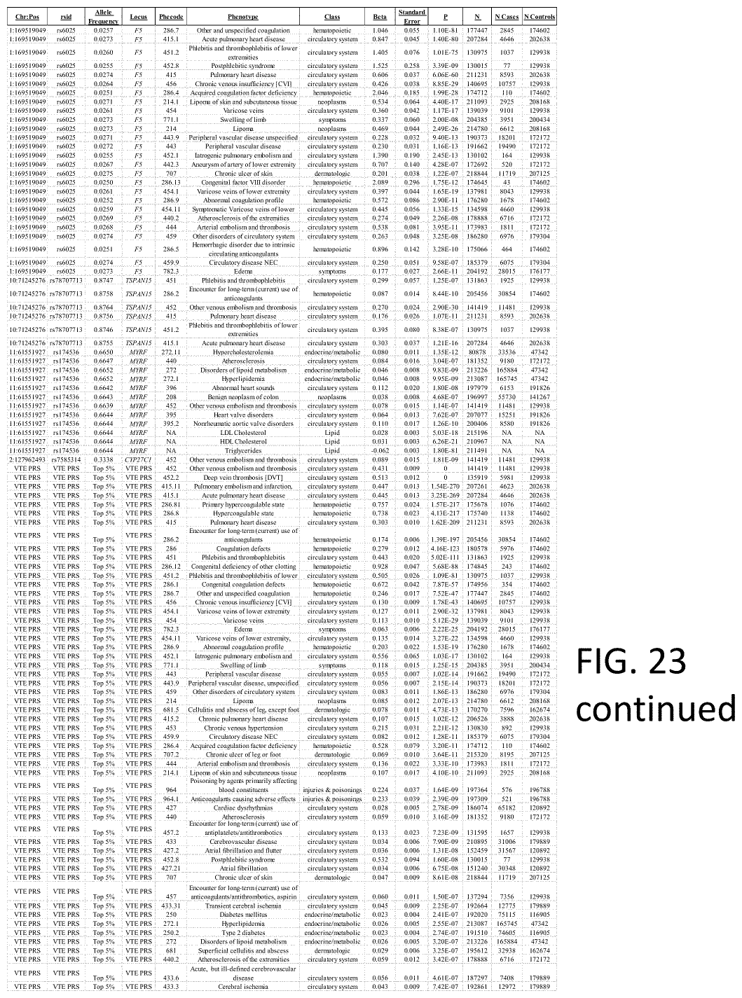

Understanding the full spectrum of phenotypic consequences of a given variant can reveal the mechanism by which a variant or gene leads to disease. Termed a phenome-wide association study (PheWAS), this approach examines the association of a risk variant across a range of phenotypes. Using a median of 63 distinct EHR-derived ICD-9/10 diagnosis codes per participant and available clinical laboratory data, each of the 30 autosomal VTE lead risk variants were tested across 1,249 disease phenotypes, symptoms, injuries, and 4 continuous cardiometabolic traits. Several of the VTE risk variants demonstrated a range of pleiotropy (FIG. 23). For example, rs2074492 near HLA-C, was associated with multiple autoimmune diseases including an increased risk for celiac disease, a disorder previously associated with a greater risk of developing VTE9. Interestingly, 4 of the VTE risk loci demonstrated known associations with LDL cholesterol (MYRF, HLA-C, ABO, and SLC44A2), and 2 with HDL (high-density lipoprotein) cholesterol/triglycerides (MYRF, PEPD). In total, 142 statistically significant (P<1.1×10−6) PheWAS associations were identified across the 30 genetic variants. Results of a PheWAS of the PRSVTE in MVP are also shown in FIG. 23.

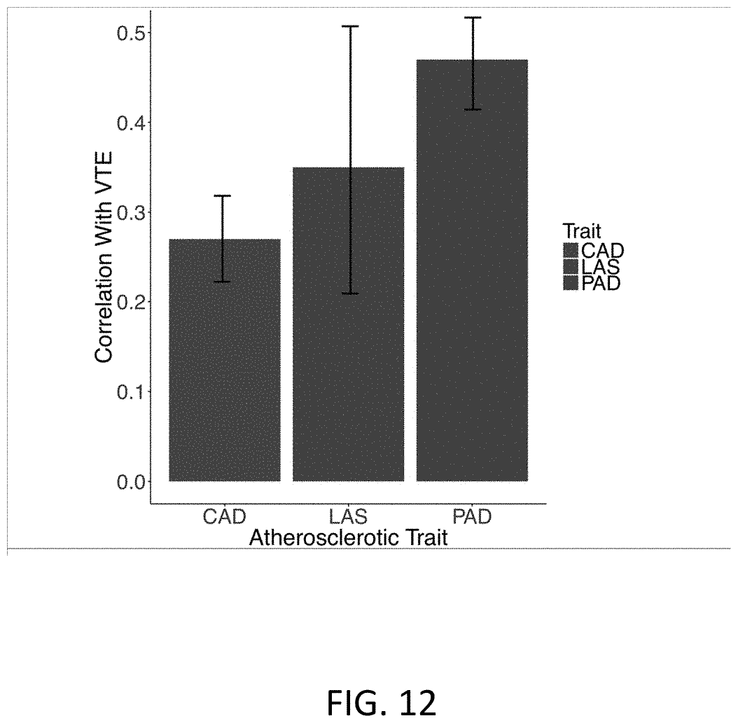

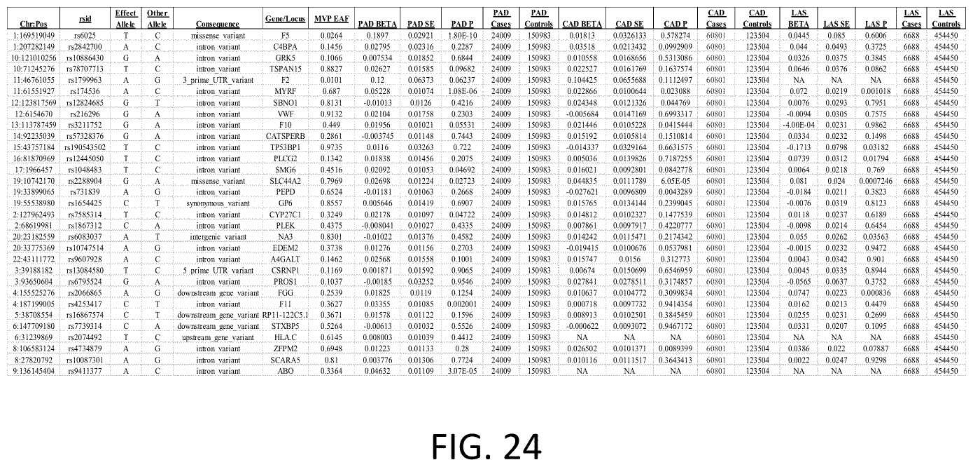

How DNA sequence variants might differ in their contribution to vascular disease risk in the arterial and venous territories was investigated. Analysis of shared heritability provides a mechanism to better understand the relationship of common variant risk across phenotypes. Using linkage disequilibrium score regression, the genetic correlation was examined between VTE and i) coronary artery disease (CAD), ii) peripheral artery disease (PAD), and iii) large artery stroke (LAS). Summary statistics was used from the European UK Biobank VTE analysis, data from a European MVP release 2.1 PAD analysis, summary data of 60,801 CAD cases and 123,504 controls from the CARDIoGRAMplusC4D consortium, and 6,688 LAS cases and 454,450 controls from the 2018 MEGASTROKE analysis. A stronger positive correlation was noted between VTE and PAD (rg=0.47, P=2.0×10⁻¹⁵) than for VTE and CAD (rg=0.27, P=1.2×10⁻⁷) or VTE and LAS (rg=0.35, P=0.02, FIG. 12), indicating that common variant risk links thrombotic complications across venous and arterial beds, but more greatly with peripheral vasculature. Using linkage disequilibrium score regression, a stronger positive correlation between VTE [N=14,222 cases; 372,102 controls] and PAD [N=24,009 cases; 150,983 controls] (r_{g}=0.47, P=2.0×10⁻¹⁵) than for VTE and CAD [N=60,801 cases; 123,504 controls] (r_{g}=0.27, P=1.2×10⁻⁷) or VTE and LAS [N=6,688 cases; 454,450 controls] (r_{g}=0.35, P=0.02) was observed across the genome. In a sensitivity analysis, the correlation between VTE and myocardial infarction (MI) was similar in direction and magnitude as that for VTE-CAD (VTE-MI rg=0.29, P=2.2×10⁻⁷). Association results for the 30 autosomal genome-wide lead VTE risk variants for PAD, CAD, and LAS in the MVP, CARDIoGRAMplusC4D, and MEGASTROKE analyses, respectively, are shown in FIG. 24.

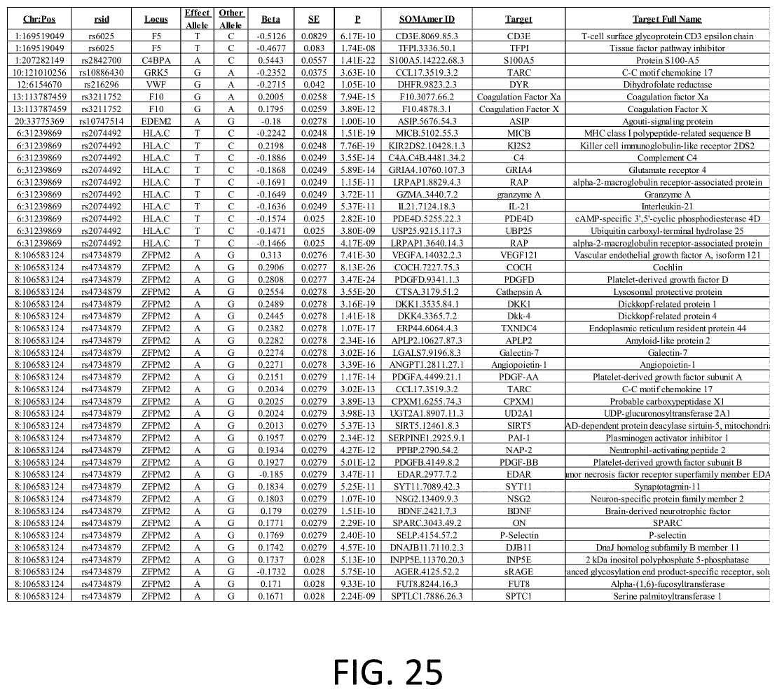

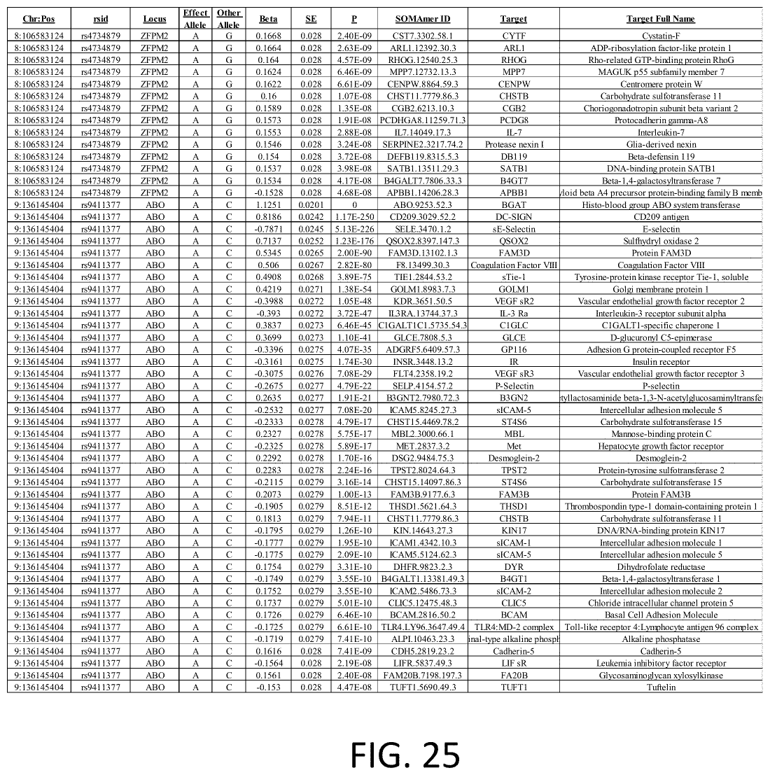

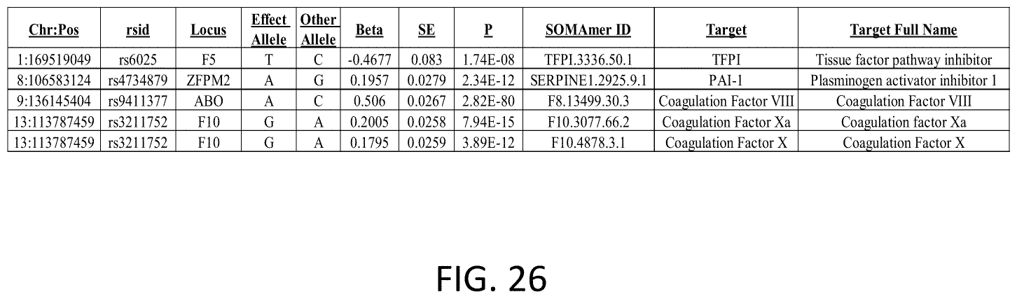

Whether the identified VTE risk variants were associated with changes in protein concentrations in circulating plasma were examined and queried recently published protein quantitative trait loci (pQTL) data derived from the plasma samples of 3,301 participants of the INTERVAL study2,17. 102 pQTL associations were observed in human plasma at genome-wide significance (P<5×10−8, FIG. 25) including 5 VTE lead variant-protein associations directly related to the coagulation cascade (FIG. 26). VTE risk alleles were associated with decreased concentration of tissue factor pathway inhibitor (TFPI), and increased concentrations of plasminogen activator-inhibitor 1 (PAI-1), Factor VIII (F8), Factor X (F10) and its active form, Factor Xa. In each case, the VTE nsk allele was associated with changes in protein concentration resulting in a pro-coagulant effect on the coagulation cascade.

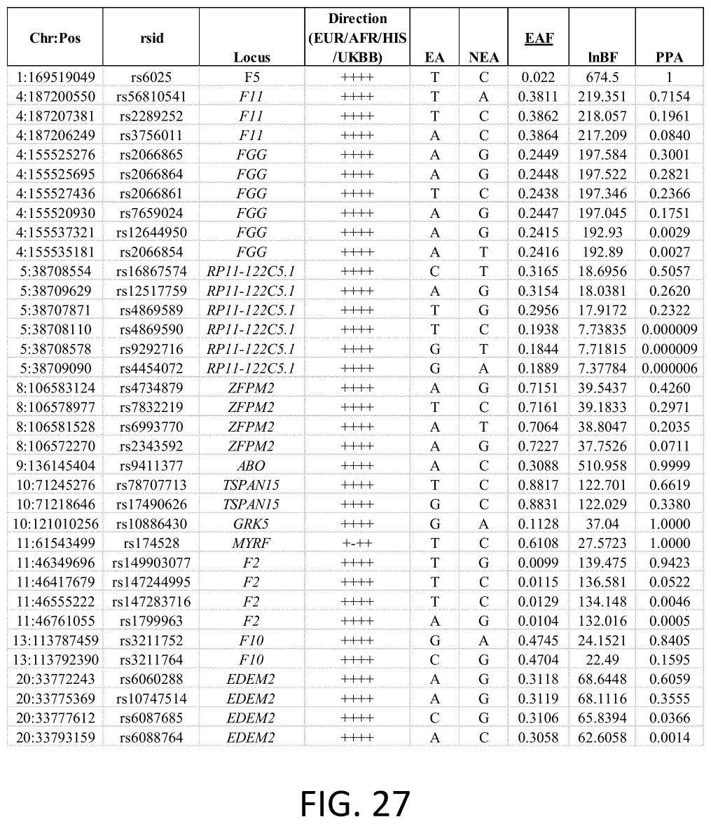

Causal VTE variants were identified through a fine-mapping analysis leveraging our multi-ethnic summary statistics and the MR-MEGA software. After excluding chromosome X and the major histocompatibility complex because of the complex LD structures across the regions, credible sets were constructed for 29 VTE loci that in aggregate account for >99% of the posterior probability of driving the VTE association based on the UK Biobank and trans-ethnic MVP summary statistics. At 12 VTE signals, the credible set included 6 or fewer VTE associated variants (FIG. 27). These credible sets included the known causal F5 Leiden19 and F2 G20210A20 variants, and also included 4 variants at the ZFPM2 locus—all of which were genome-wide trans-pQTL associations with PAI-1 (FIG. 18).

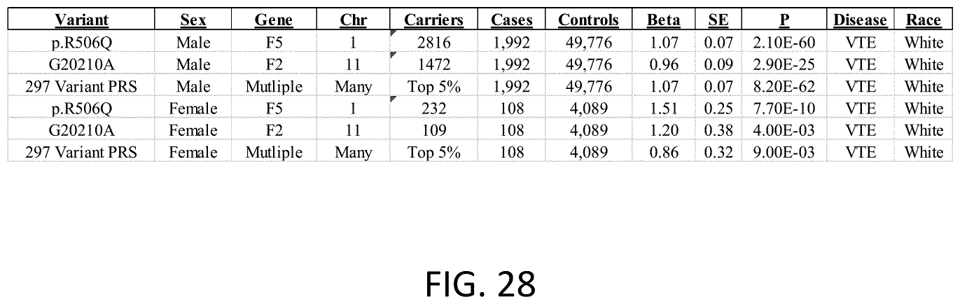

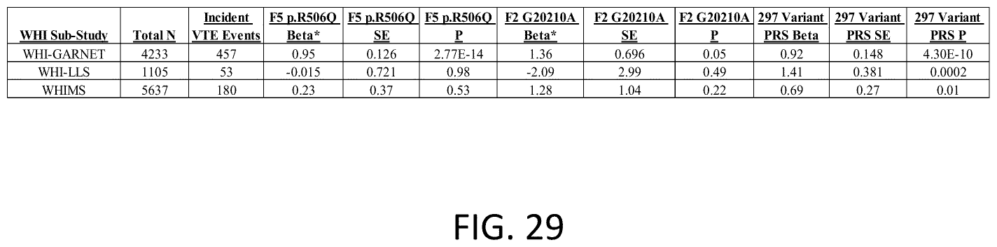

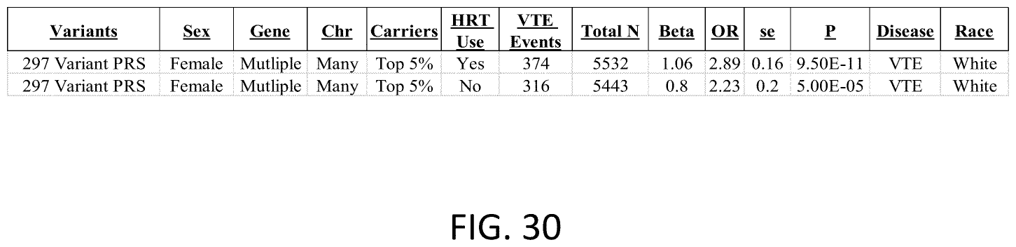

For the VTE PRS analysis, MVP release 3.0 results stratified by sex were provided in FIG. 28, and results of the incident event analysis in WHI stratified by WHI sub-study as well as by hormone replacement therapy use are shown in FIGS. 29-30.

Lastly, in MVP a manual chart review of 50 VTE cases and 50 controls were performed, which demonstrated that our phenotyping algorithm had a positive predictive value of 96% (95% CI=85.1-99.3%), and negative predictive value of 100% (95% CI=91.1-100%).

2. Methods

i. Study Populations

Genetic association analyses were conducted using DNA samples and phenotypic data from two cohorts: the Million Veteran Program (MVP) and UK Biobank. In MVP, individuals aged 19 to over 100 years were recruited from 63 VA Medical Centers across the United States. In the initial MVP analysis, 11,844 VTE cases (8,929 white, 2,261 black, 654 Hispanic) and 211,753 VTE-free controls were evaluated.

In UK Biobank, individuals aged 45 to 69 years old were recruited from across the United Kingdom for participation. In this study, 14,222 VTE cases and 372,102 controls of European ancestry were identified.

In addition, incident VTE data was examined from the WHI randomized clinical trial of Hormone Therapy (HT) for the PRS analysis. In brief, at the inception of the WHI study (1993-1998), 161,808 postmenopausal women between the ages of 50 and 79 years were eligible for inclusion in multiple clinical trials. Exclusion criteria related to the presence of medical conditions predisposing to shortened survival or safety concerns. The protocol and consent forms were approved by institutional review committees and all participants provided written informed consent. The WHI-HT initially comprised 27,347 postmenopausal women who were randomized to receive either estrogen plus progestin or estrogen alone versus placebo until the trials were stopped early in July 2002 and March 2004, respectively. All WHI-HT participants subsequently continued to be followed without intervention until close-out. Of the various components of WHI, VTE was adjudicated by physician adjudicators for participants who were enrolled in the HT trials.

ii. Genetic Data and Quality Control for Association Analysis

DNA extracted from whole blood was genotyped in MVP using a customized Affymetrix Axiom biobank array, the MVP 1.0 Genotyping Array. Veterans (U.S. military personnel) of three mutually exclusive ethnic groups were identified for analysis: 1) non-Hispanic whites (European ancestry), 2) non-Hispanic blacks (African ancestry), and 3) self-identified Hispanics. After pre-phasing using EAGLE33 v2, genotypes from the 1000 Genomes Project phase 3, version 5 reference panel were imputed into MVP participants via Minimac3 software. Ethnicity-specific principal component analysis was performed using the EIGENSOFT software.

In MVP, sample and variant quality control was performed as previously described36. In brief, duplicate samples, samples with more heterozygosity than expected, an excess (>2.5%) of missing genotype calls, or discordance between genetically inferred sex and phenotypic gender were excluded. In addition, one individual from each pair of related individuals (kinship>0.0884 as measured by the KING37 software) were removed. In total, 312,571 multi-ethnic participants passing quality control from the MVP release 2.1 data (used in association analysis) were identified, and another 69,578 from the MVP release 3.0 data used for the PRS analysis.

Following imputation, variant-level quality control was performed using the EasyQC R package and exclusion metrics included: ancestry-specific Hardy-Weinberg equilibrium P<1×10−20, posterior call probability <0.9, imputation quality <0.3, minor allele frequency (MAF)<0.0003, call rate <97.5% for common variants (MAF>1%), and call rate <99% for rare variants (MAF<1%). Variants were also excluded if they deviated >10% from their expected allele frequency based on reference data from the 1000 Genomes Project. Following variant-level quality control, 19.9 million, 31.9 million, and 28.1 million DNA sequence variants were obtained for analysis in white, black, and Hispanic participants, respectively.

In UK Biobank, analysis was performed separately in white individuals after genotyping using either the UK BiLEVE or UK Biobank Axiom Arrays. Approximately 500,000 individuals were genotyped and subsequently imputed to the haplotype reference consortium (HRC) and UK10K reference panels (UK Biobank v3 release). Genome-wide association testing was performed for VTE in the UK Biobank using all variants in the v3 release with minor allele frequency greater than 0.3% and imputation quality INFO >0.4. To avoid potential population stratification, only European-ancestry samples were included in the analysis. This subset was selected based on self-reported white ethnicity that was subsequently confirmed using genetic principal components analysis. Outliers within the self-reported white samples in the first 6 principal components of ancestry were detected and subsequently removed using the R package aberrant. In addition, individuals with sex chromosome aneuploidy (neither XX or XY), discordant self-reported and genetic sex, or excessive heterozygosity or missingness, as defined centrally by the UK Biobank were removed. Finally, one individual from each pair of second-degree or closer relatives (kinship >0.0884) was removed, selectively retaining VTE cases when possible.

iii. VTE Discovery Association Analysis

In MVP, genotyped and imputed DNA sequence variants were tested for association with VTE through logistic regression adjusting for age, sex, and 5 principal components of ancestry assuming an additive model using the SNPTEST (mathgen.stats.ox.ac.uk/genetics software/snptest/snptest.html) statistical software program. In the discovery analysis, association analyses were performed using MVP release 2.1 data separately for each ancestral group (whites, blacks, and Hispanics) and then meta-analyzed using an inverse-variance weighted fixed effects method implemented in the METAL software program. Variants with a high amount of heterogeneity (12 statistic >75%) across the three ancestries were excluded. In UK Biobank, association testing was performed using a logistic regression model adjusted for age at baseline, sex, genotyping array, and the first 5 principal components of ancestry. All testing was performed in PLINK2 (.cog-genomics.org/plink/2.0/).

Results were combined across MVP release 2.1 and UK Biobank cohorts using inverse-variance weighted fixed effects meta-analysis and set a significance threshold of P<5×10⁻⁸ (genome-wide significance). In addition, an internal replication P<0.01 was required in each of the MVP and UK Biobank analyses (e.g. MVP discovery and subsequent UK Biobank replication, and vice versa), with concordant direction of effect, to minimize false positive findings. Novel loci were defined as being greater than 500,000 base-pairs away from a known VTE genome-wide associated lead variant. Additionally, European linkage disequilibrium information from the 1000 Genomes Project was used to determine independent variants where a locus extended beyond 500,000 base-pairs. All logistic regression P values were two-sided. For X chromosome analyses, male genotypes were coded as if they were homozygous diploid for the observed allele.

iv. Replication

In Phase 2, an additional round of external replication was performed for lead variants using summary data of up to 15,572 VTE cases and 113,430 disease-free controls from the INVENT consortium's current VTE meta-analysis combined with 2,100 VTE cases and 53,865 controls from MVP 3.0 data. Of note, UK Biobank data was excluded from the summary statistics provided by INVENT. Significant novel associations were defined as those that were at least nominally significant in replication (P<0.05) with consistent direction of effect and had an overall P<5×10⁻⁸ (genome-wide significance) in the discovery and replication cohorts combined.

v. Venous Thromboembolism Disease Definitions

From the 312,571 multi-ethnic participants in MVP release 2.1, and 69,578 European participants in MVP release 3.0, individuals were defined as having VTE based on possessing at least two of the ICD-9/10 codes outlined in Table 5 in their EHR. Individuals were defined as controls if did not meet the definition of a VTE case and their EHR reflected 2 or more separate encounters in the Veterans Affairs Healthcare System in each of the two years prior to enrollment in MVP.

TABLE 5
ICD9/10 Diagnosis codes used for MVP venous thromboembolism definition

| Disease | Code | Description | Coding System |
|---|---|---|---|
| Deep Venous | I80.1** | Phlebitis and thrombophlebitis of the femoral vein | ICD-10-CM |
| Thrombosis | I80.2** | Phlebitis and thrombophlebitis of other and | ICD-10-CM |
| | | unspecified deep vessels of the lower extremities | |
| | I82.22 | Embolism and thrombosis of inferior vena cava | ICD-10-CM |
| | I82.4** | Acute embolism and thrombosis of deep veins of | ICD-10-CM |
| | | lower extremity. | |
| | I82.5** | Chronic embolism and thrombosis of deep veins | ICD-10-CM |
| | | of lower extremity | |
| | 451.11 | Phlebitis and thrombophlebitis of femoral vein | ICD-9-CM |
| | | (deep) (superficial) | |
| | 451.19 | Phlebitis and thrombophlebitis of deep veins of | ICD-9-CM |
| | | lower extremities, other | |
| | 453.2 | Phlebitis and thrombophlebitis of lower | ICD-9-CM |
| | | extremities, unspecified | |
| | 453.4 | Acute venous embolism and thrombosis of deep | ICD-9-CM |
| | | vessels of lower extremity. | |
| Pulmonary | I26.0** | Pulmonary embolism with acute cor pulmonale | ICD-10-CM |
| Embolism | I26.9** | Pulmonary embolism without acute cor | ICD-10-CM |
| | | pulmonale | |
| | 415.1 | Pulmonary embolism and infarction | ICD-9-CM |

vi. Lipids and VTE Mendelian Randomization Analysis

Summary-level data for 222 genome-wide lipids-associated variants were obtained from the publicly available data from the Global Lipids Genetics Consortium using a previously described genetic risk score instrument. Cohorts either excluded participants on statins or adjusted total cholesterol and LDL cholesterol (by dividing by 0.8 or 0.7, respectively) if a statin was prescribed. One variant, rs77375493, was excluded from the current analysis after not passing quality control. Results were utilized from the MVP and UK Biobank GWAS meta-analysis restricted to Europeans. The effect alleles were matched with all lipid and VTE summary data and 3 different Mendelian randomization analyses were performed: 1) inverse-variance weighted; 2) multivariable; 3) MR-Egger to account for pleiotropic bias. First, inverse-variance weighted Mendelian randomization was performed using each set of variants for each lipid trait as instrumental variables. This method, however, does not account for possible pleiotropic bias. Therefore, inverse-variance weighted multivariable Mendelian randomization was then performed. This method adjusts for possible pleiotropic effects across the included lipid traits in our analyses using effect estimates from the variant-VTE outcome and effect estimates from variant-LDL cholesterol, variant-HDL cholesterol, and variant-triglycerides as predictors in 1 multivariable model. MR-Egger was additionally performed. This technique can be used to detect bias secondary to unbalanced pleiotropy in Mendelian randomization studies. In contrast to inverse-variance weighted analysis, the regression line is unconstrained, and the intercept represents the average pleiotropic effects across all variants. Bonferroni-corrected 2-sided P values (P=0.016; 0.05/3) for 3 tests were used to declare statistical significance. Analysis was performed using the R software program.

vii. Colocalization of ZFPM2 VTE GWAS and PAI-1 Plasma pQTL Signals

To evaluate whether there was evidence of colocalization across the VTE GWAS and PAI-1 pQTL studies, European MVP release 2.1 and UK Biobank European VTE meta-analyzed summary statistics and PAI-1 pQTL results from the INTERVAL study were used. For the 2,178 variants within the 1-megabase region surrounding the lead ZFPM2 lead VTE GWAS variant, a locus-wide colocalization analysis was performed using FINEMAP42 to generate posterior causal probabilities for each of these variants in the GWAS and the pQTL analyses. The European superpopulation subset of the 1000 Genomes phase 3 whole genome sequence data was used as a reference for the linkage disequilibrium statistics, and assumed only 1 causal variant at the locus. These posterior probabilities were analyzed with a publicly available pipeline to compute the CLPP for the entire locus. The R package LocusCompareR was used to visualize the colocalizing signals.

viii. Functional Assessment of PAI-1 in Murine Models

Male C57BL/6 (WT) mice (Jackson Laboratory, Farmington, Conn.), PAI-1−/− (backcrossed 5-10 generations on C57BL/6 mice) and PAI-1 over-expressing mice (PAI-1 Tg, backcrossed 5-10 generations on C57BL/6 background) were utilized in this study. Previous data comparing homozygous littermates to wild type C57BL/6 controls demonstrated identical phenotype with regards to venous thrombosis with regards to size and cellular composition. Therefore, in the interest of humane and responsible animal use, wild type C57BL/6 mice (WT) were utilized as controls. Animals underwent a well-characterized DVT model, stasis inferior vena cava (IVC) thrombosis, at 8-10 weeks of age and 20-25 grams body weight. Isoflurane 2% was administered as inhaled anesthetic. A midline laparotomy was performed, the retroperitoneum exposed, and dorsal IVC branches were interrupted with electrocautery. The infrarenal IVC and any accompanying side branches caudal to the left renal vein were ligated with 7-0 prolene (Ethicon, Inc., Somerville, N.J.) to generate blood stasis. A running continuous 5-0 Vicryl suture was used to close the fascia and Vetbond tissue adhesive was applied for skin closure (3M Animal Care Products, St. Paul, Minn.). Mice were euthanized at 6 and 14 days post-thrombosis. The IVC and its associated thrombus were weighed (grams) and measured (centimeters) for weight to length analysis of thrombus size. GraphPad Prism software version 6.0 was used to analyze the thrombus size. Data is presented as the mean +/− the standard deviation. Statistical significance amongst multiple groups was determined using one-way analysis of variance followed by Tukey's multiple comparisons post hoc test. A value of P<0.05 was considered significant.

ix. VTE Polygenic Risk Score Generation

Polygenic risk scores (PRS) represent an individual's risk of a given disease conferred by the cumulative impact of many common DNA sequence variants. A weight is assigned to each genetic variant based on its strength of association with disease risk (β). Individuals are then additively scored in a weighted fashion based on the number of risk alleles they carry for each variant in the PRS.

To generate the score, summary statistics was used from the combined MVP release 2.1 and UK Biobank VTE summary statistics restricted to Europeans (23,151 VTE cases, 553,439 controls) and a linkage disequilibrium panel of 20,000 randomly selected European samples from UK Biobank. Variants were restricted to those present in both MVP release 2.1 and UK Biobank VTE summary statistics with a consistent direction of effect. To increase the number of independent variants included in the score, a pruning and thresholding analysis was performed using the linkage disequilibrium-driven clumping procedure in PLINK version 1.90b (--clump). In brief, this algorithm formed “clumps” around variants with VTE association P<1×10⁻⁵ and with an R2>0.2 based on the linkage disequilibrium reference. From our initial set of summary statistics, the algorithm selects only 1 associated variant from each clump below our pre-specified P value threshold. The final output from this procedure generated a score of 299 independent (R2<0.2), VTE associated (P<1×10⁻⁵) variants, representing the strongest disease-associated variant for each linkage disequilibrium-based clump across the genome. From this 299 variant PRS, the clumps containing the F5 p.R506Q and F2 G20210A variants were then removed, resulting in a 297 variant PRSVTE for downstream analysis.

x. VTE Polygenic Risk Score Analysis

From the 69,578 MVP release 3.0 participants (none of whom were included in the VTE discovery analysis), 2,100 prevalent VTE cases and 53,865 controls were identified. The associated VTE nsk was assessed for the 5% of individuals with the highest PRSVTE relative to the rest of the population using logistic regression adjusting for age, sex, and 5 principal components of ancestry. The association of the F5 p.R506Q and F2 G20210A variants were then tested among the 5% of individuals with the highest PRSVTE relative to the rest of the population in the MVP release 3.0 data using an identical logistic regression model.

The findings were replicated using incident VTE data from the WHI. Data used in this analysis included genetic data from WHI-HT participants derived from three separate GWAS sub-studies: 1) the WHI Genomics and Randomized Trials Network study (WHI-GARNET, 457 incident VTE events among 4,233 participants), (2) the WHI Memory Study (WHIMS, 180 incident VTE events among 5,637 participants), and (3) the WHI Long Life Study (WHI-LLS, 53 incident VTE events among 1,105 participants). All individuals included in the incident event analysis were of European ancestry. Specific details of each WHI sub-study including genotyping, study design, and imputation are included in the Supplementary Note. Cox proportional hazards models were used to estimate hazard ratios (HR) and 95% confidence intervals for the associations of the F5 p.R506Q and F2 G20210A mutations with VTE adjusting for age, 10 principal components of ancestry, and hormone therapy intervention status during the active phase of the WHI-HT. The associated VTE risk for the 5% of individuals with the highest PRSVTE relative to the rest of the population was then tested using Cox proportional hazards models adjusting for age, 10 principal components of ancestry, and hormone therapy intervention status during the active phase of the WHI-HT. Results from WHIMS, WHI-LLS, and WHI-GARNET were combined using an inverse-variance weighted fixed effects meta-analysis. Bonferroni-corrected 2-sided P values (P=0.016; 0.05/[2 variants+1 PRSVTE) for 3 tests were used to declare statistical significance. Analyses were performed using the R software program (version 3.2.1; Vienna, Austria).

xi. Data Availability

The full summary level association data from the MVP trans-ancestry PAD meta-analysis from this report are available through dbGAP, accession code phs001672.v2.pl. Data contributed by CARDIoGRAMplusC4D investigators are available online (http://www.CARDIOGRAMPLUSC4D.org/). Data on large artery stroke have been contributed by the MEGASTROKE investigators and are available online (http://www.megastroke.org/). The genetic and phenotypic UK Biobank data are available upon application to the UK Biobank.

xii. Cohort Descriptions

The design of the Million Veteran Program (MVP) was previously described. In brief, individuals aged 19 to >100 years have been recruited from more than 50 VA Medical Centers nationwide since 2011. Each veteran's electronic health record (EHR) data are being integrated into the MVP biorepository, including inpatient International Classification of Diseases (ICD-9/10) diagnosis codes, Current Procedural Terminology (CPT) procedure codes, clinical laboratory measurements, and reports of diagnostic imaging modalities. MVP received ethical and study protocol approval by the VA Central Institutional Review Board and informed consent was obtained from all participants.

In UK Biobank, individuals aged 45 to 69 years old were recruited from across the United Kingdom for participation. At enrollment, a trained healthcare provider ascertained participants' medical histories through verbal interview. In addition, participants' EHR including inpatient ICD-9/10 diagnosis codes and Office of Population and Censuses Surveys (OPCS-4) procedure codes, were integrated into UK Biobank. Informed consent was obtained for all participants, and UK Biobank received ethical approval from the Research Ethics Committee (reference number 11/NW/0382). Our study was approved by a local Institutional Review Board at Partners Healthcare (protocol 2013P001840).

Incident VTE data from the Women's Health Initiative (WHI) randomized clinical trial of Hormone Therapy (HT) was analyzed for the PRS analysis. In brief, at the inception of the WHI study (1993-1998), 161,808 postmenopausal women between the ages of 50 and 79 years were eligible for inclusion in multiple clinical trials. Exclusion criteria related to the presence of medical conditions predisposing to shortened survival or safety concerns. The protocol and consent forms were approved at each site by institutional review committees and all participants provided written informed consent. The WHI-HT initially comprised 27,347 postmenopausal women who were randomized to receive either estrogen plus progestin or estrogen alone versus placebo until the trials were stopped early in July 2002 and March 2004, respectively. All WHI-HT participants subsequently continued to be followed without intervention until close-out. Of the various components of WHI, VTE was adjudicated by physician adjudicators for participants who were enrolled in the HT trials. The WHI-HT trial was approved by the local institutional review board at the Fred Hutchinson Cancer Research Center.

xiii. Quality Control Analysis

In MVP, the following were excluded: duplicate samples, samples with more heterozygosity than expected, an excess (>2.5%) of missing genotype calls, or discordance between genetically inferred sex and phenotypic gender. In addition, one individual from each pair of related individuals (as measured by the KING software) were removed. Veterans were then divided into three mutually exclusive ethnic groups based on self-identified race/ethnicity and admixture analysis using the ADMIXTURE v1.3 software: 1) non-Hispanic whites (self-identified as “non-Hispanic,” “white,” and >80% genetic European ancestry), 2) non-Hispanic blacks (self-identified as “non-Hispanic,” “black,” and >50% genetic African ancestry), and 3) Hispanics (self-identified only). In total, 312,571 white, black, and Hispanic MVP participants passed our sample-level quality control. Prior to imputation, variants that were poorly called or that deviated from their expected allele frequency based on reference data from the 1000 Genomes Project were excluded. After pre-phasing using EAGLE v2, genotypes from the 1000 Genomes Project phase 3, version 5 reference panel were imputed into Million Veteran Program (MVP) participants via Minimac3 software. Ethnicity-specific principal component analysis was performed using the EIGENSOFT software.

Following imputation, variant level quality control was performed using the EasyQC R package (www.R-project.org), and exclusion metrics included: ancestry specific Hardy-Weinberg equilibrium P<1×10⁻²⁰, posterior call probability <0.9, imputation quality <0.3, minor allele frequency (MAF)<0.003, call rate <97.5% for common variants (MAF>1%), and call rate <99% for rare variants (MAF<1%). Variants were also excluded if they deviated >10% from their expected allele frequency based on reference data from the 1000 Genomes Project.

In UK Biobank, approximately 500,000 individuals were genotyped and subsequently imputed to the haplotype reference consortium (HRC) and UK10K reference panels. Genome-wide association testing was performed for VTE in the UK Biobank using all variants in the v3 release with minor allele frequency greater than 0.3% and imputation quality INFO >0.4. To avoid potential population stratification, only European-ancestry samples were included in the analysis. This subset was selected based on self-reported white ethnicity that was subsequently confirmed using genetic principal components analysis. Outliers within the self-reported white samples in the first 6 principal components of ancestry were detected and subsequently removed using the R package aberrant. In addition, individuals with sex chromosome aneuploidy (neither XX or XY), discordant self-reported and genetic sex, or excessive heterozygosity or missingness, as defined centrally by the UK Biobank were removed. Finally, one individual from each pair of second-degree or closer relatives (kinship >0.0884) was removed, selectively retaining VTE cases when possible.

xiv. VTE Phenotype and Manual Chart Review

Manual chart review was performed by two blinded trained clinician chart abstractors with a vascular surgeon reviewing discordant cases; the results of chart review for 50 cases and 50 controls otherwise representative of the overall cohort were used for determining the positive and negative predictive values of the phenotyping algorithm, which was standardized to the 50% prevalence of VTE in the validation set. Positive predictive value refers to the ratio of (true positives)/(true positives+false positives) and negative predictive value the ratio of (true negatives)/(true negatives+false negatives). In UK Biobank, individuals were defined as having VTE based on the definition by Klarin and colleagues as previously described. All other individuals were defined as controls.

xv. Discovery and Replication Association Analysis

In MVP, genotyped and imputed DNA sequence variants were tested for association with VTE through logistic regression adjusting for age, sex, and 5 principal components of ancestry assuming an additive model using the SNPTEST (mathgen.stats.ox.ac.uk/genetics software/snptest/snptest.html) statistical software program. In the discovery analysis, association analyses was performed separately for each ancestral group (whites, blacks, and Hispanics) and then meta-analyzed using an inverse variance-weighted fixed effects method implemented in the METAL software program. Variants with a high amount of heterogeneity (I² statistic >75%) across the three ancestries were excluded.

In UK Biobank, association testing was performed using a logistic regression model adjusted for age at baseline, sex, genotyping array, and the first 5 principal components of ancestry. All testing was performed in PLINK2 (https://www.cog-genomics.org/plink/2.0/). Results were combined across MVP and UK Biobank cohorts using inverse-variance weighted fixed effects meta-analysis and set a significance threshold of P<5×10⁻⁸ (genome-wide significance). In addition, a replication P<0.01 in each of the MVP and UK Biobank analyses (e.g. MVP discovery and subsequent UK Biobank replication, and vice versa) were required, with concordant direction of effect, to minimize false positive findings. Novel loci were defined as being greater than 500,000 base-pairs away from a known VTE genome-wide associated lead variant. Additionally, linkage disequilibrium information from the 1000 Genomes Project was used to determine independent variants where a locus extended beyond 500,000 base-pairs. All logistic regression P values were two-sided.

xvi. Conditional Analysis

The COJO-GCTA software was used to perform an approximate, stepwise conditional analysis to identify independent variants within VTE-associated loci. Summary statistics were used from the European specific meta-analysis of UK Biobank and MVP datasets (23,151 VTE cases, 553,439 controls) to conduct this analysis combined with an LD-matrix obtained from 20,000 unrelated European individuals randomly sampled from the UK Biobank release v3. A threshold P<5×10⁻⁸ (genome-wide significance) was set to declare statistical significance.

xvii. PheWAS Disease Definitions, and Association Analysis

Understanding the full spectrum of phenotypic consequences of a given DNA sequence variant may shed light on the mechanism by which a variant/gene leads to disease. For 30 autosomal lead VTE risk variants and the PRS_{VTE} identified in the study, a PheWAS was performed of 1,249 distinct diseases, symptoms, and injuries in MVP leveraging the full catalog of EHR ICD-9/10 diagnosis codes in 227,817 white veterans using the R package PheWAS. 4 continuous cardiometabolic traits—LDL cholesterol, HDL cholesterol, triglycerides, and body mass index—were also used given their possible links with VTE causality. In total, 1,249 disease phenotypes and 4 continuous traits were available for analysis and a statistical threshold of P<1.2×10⁻⁶ [0.05/(31×(1,249 diseases+4 continuous traits))] was set. Of 312,571 genotyped veterans passing quality control, 23,172,451 distinct, prevalent ICD-9 diagnosis codes available were performed for analysis. The largest ethnic group of 227,817 white participants was focused on, in which the mean age was 64.3±13.1 years, and 93.3% (212,465) were male.

ICD-9 diagnosis codes were collapsed to clinical disease groups and corresponding controls using the groupings proposed by Denny et al. Diseases were required to have a prevalence of >0.13% (˜300 cases) to be included in the PheWAS analysis. 30 autosomal lead VTE risk DNA sequence variants and the PRS_{VTE} were tested using logistic regression adjusting for age, sex, and five principal components under the assumption of additive effects using the PheWAS R package (github.com/PheWAS/PheWAS) in R v3.2.0 (.R-project.org). In total, 1,249 disease phenotypes and 4 continuous traits were available for analysis and a statistical threshold of P<1.1×10⁻⁶ [0.05/(31×(1,249 diseases+4 continuous traits))] was set. For the lipid continuous traits (LDL cholesterol, HDL cholesterol, and triglycerides), maximal LDL cholesterol/triglycerides (after log transformation) and minimal HDL cholesterol were used after inverse normal transformation in MVP. The body-mass index (BMI) phenotype was formulated in both UK Biobank and MVP and results were combined in an inverse-variance weighted fixed effects meta-analysis. In UK Biobank, BMI was calculated in 374,942 unrelated individuals from the measurement acquired at enrollment. In MVP, BMI was calculated in 218,382 participants from the mean height and mean weight over the 3 years prior to the enrollment date. Outliers were excluded if their mean measurement was <17 or >60 kg/m². In each case, the BMI phenotype was adjusted for age, age squared, and principal components of ancestry in a linear regression model. The resulting residuals were transformed to approximate normality using inverse normal scores separately by sex. All logistic and linear regression P values were two-sided.

xviii. Shared Heritability within PAD, CAD, and LAS

To better understand the how common genetic variation influences risk for atherosclerosis in multiple vascular beds, linkage disequilibrium score regression was used to calculate the genetic correlation between VTE-PAD, VTE-CAD/VTE-MI and VTE-LAS. Summary statistics from the European UK Biobank VTE GWAS, European MVP PAD GWAS, the CARDIoGRAMplusC4D CAD/MI GWAS (predominantly European), and the transancestral LAS MEGASTROKE GWAS meta-analysis (>2/3 European) were used for this analysis. Of note, the transancestral meta-analysis statistics were used from MEGASTROKE because the sample size of the European-ancestry only analysis lacked sufficient power for estimation of genetic correlation. Association results were queried for the 30 autosomal genome-wide lead VTE risk variants for PAD, CAD, and LAS in the MVP PAD, CARDIoGRAMplusC4D CAD, and MEGASTROKE LAS GWAS analyses, respectively.

xix. VTE Variant-Plasma Protein Associations

To identify loci that might influence plasma protein concentrations potentially implicated in thromboembolism, published protein quantitative trait locus (pQTL) data generated from an aptamer-based multiplex protein assay was used to quantify 3,622 plasma proteins in 3,301 healthy participants from the INTERVAL study. The 30 autosomal VTE risk variants identified in the study were quantified for overlap with genome-wide significant (two-sided P<5.0×10⁻⁸) variant-protein pairs.

xx. Fine-Mapping of VTE Association Signals

For 29 non-MHC, autosomal VTE association signals, the MR-MEGA software and VTE summary statistics were used from the UK Biobank (European) and MVP (African, European, and Hispanic) analyses. A genomic region 1 mega-base on either side of the VTE lead variant restricting to variants with MAF>1% was verified. Under the assumption of one causal variant at a given locus, multi-dimensional scaling of the Euclidean distance matrix was then used to generate axes of genetic variation to each set of association statistics between ancestry groups as implemented in MR-MEGA. For each GWAS signal the “meta-regression model,” including one axis of genetic variation as a covariate, was applied to each variant passing quality control. From this model, the VTE association was examined for each variant and the heterogeneity in allelic effects that is correlated with ancestry. Subsequently, a posterior probability (using the resultant Bayes' factor) of VTE association was derived and a 99.99% credible set of variants was constructed driving each GWAS signal.

xxi. Genetic Analysis of Incident VTE Events in WHI

After assessing the associated VTE risk for F5 p.R506Q and F2 G20210A carriers as well as the 5% of individuals with the highest PRS_{VTE} relative to the rest of the population in MVP, replication of the findings was sought using incident VTE data from the WHI. In brief, at the inception of the WHI study postmenopausal women between the ages of 50 and 79 years were eligible for inclusion in multiple clinical trials. Data used in this analysis included incident VTE events from participants belonging to one of three GWAS sub-studies: 1) the WHI Genomics and Randomized Trials Network (WHI-GARNET, 457 incident VTE events among 4,233 participants), 2) the WHI Memory Study (WHIMS, 180 incident VTE events among 5,637 participants), 3) the WHI Long Life Study (WHI-LLS, 53 incident VTE events among 1,105 participants).

The WHI-GARNET sub-study consisted of individuals selected as a nested case-control sample of coronary heart disease, stroke, venous thrombosis, and incident diabetes events from the parent WHI Hormone Therapy Trial. From 27,347 women who participated in the Hormone Therapy Trial, 4,894 were genotyped on the Illumina Omni-Quad as part of WHI GARNET and imputed using the 1000 Genomes reference panel phase 3, version 5. VTE cases were identified that occurred during the active phase of the Hormone Trial and afterwards. Controls were participants in the Hormone Therapy Trial free of all 4 case conditions. Matching criteria for controls were age, race/ethnicity, hysterectomy status, and enrollment date. GARNET WHI participants were predominantly European (87%), and only European individuals were included in the analysis. In total, 457 VTE incident events were identified among 4,233 individuals after removing 21 observations due to missingness.

The WHIMS sub-study consisted of WHI Hormone Trial women of European ancestry from the following sources: 1) WHI Memory Study (WHIMS) participants who were not in WHI-GARNET, 2) women from the WHI-HT at least 65 years old at enrollment who were neither in WHIMS nor GARNET, and 3) women from the WHI-HT younger than age 65 at enrollment who were neither in WHIMS nor GARNET. In total, 180 incident VTE events were identified among 5,637 individuals after removing 50 observations due to missingness. Participants were genotyped using the Illumina HumanOmniExpress platform and imputed using the 1000 Genomes reference panel phase 3, version 5.

The WHI-LLS (GWAS) sub-study consisted of the phase III cohort of additional eligible women who were added to the LLS study after the decision was made to expand the study population in 2012. In total, 53 VTE incident events were identified among 1,105 individuals after removing 13 observations due to missingness. Participants were genotyped using the Illumina HumanOmniExpress platform and imputed using the 1000 Genomes reference panel phase 3, version 5.

Cox proportional hazards models were used to estimate hazard ratios (HR) and 95% confidence intervals for the associations of the F5 p.R506Q and F2 G20210A mutations with VTE adjusting for age, 10 principal components of ancestry, and hormone therapy intervention status during the active phase of the WHI-HT. The associated VTE risk for the 5% of individuals with the highest PRS_{VTE} relative to the rest of the population was tested using Cox proportional hazards models adjusting for age, 10 principal components of ancestry, and hormone therapy intervention status during the active phase of the WHI-HT. Results from WHIMS, WHI-LLS, and WHI-GARNET were combined using an inverse-variance weighted fixed effects meta-analysis. Bonferroni-corrected 2-sided P values (P=0.016; 0.05/3) for 3 tests were used to declare statistical significance. Analyses were performed using the R software program (version 3.5.1; Vienna, Austria).

### 3. Discussion

These findings permit several conclusions. First, the results lend human genetic support to LDL cholesterol lowering as a preventive strategy for VTE. In the JUPITER (Justification for the Use of statins in Prevention: an Interventional Trial Evaluating Rosuvastatin) trial, administration of 20 mg of rosuvastatin in asymptomatic participants resulted in a reduced occurrence of symptomatic VTE. This implies that the apparent VTE risk reduction from statins may be due to on-target lowering of lipoproteins, much like the benefits observed for multiple atherosclerotic syndromes. Second, partial antagonism of PAI-1 as a preventive treatment for VTE deserves further consideration. Colocalizing ZFPM2 VTE GWAS and PAI-1 pQTL associations and observed PAI-1 overexpressing mice had 1.5-fold larger thrombus size compared to PAI-1−/− mice in an inferior vena cava ligation model. These results indicate that imbalance in the thrombosis-fibrinolysis pendulum in the human condition can lead to development of pathologic VTE, whereas lower active PAI-1 levels can allow for resolution of incidental venous thrombosis prior to becoming clinically relevant. Third, the data provide further evidence for the utility of polygenic risk prediction in the clinical realm. In a recent publication, the authors generate expanded PRS, and demonstrate that those within the right tail of the distribution have a >3-fold increased risk of developing the disease, akin to carriers of monogenic mutations. These findings are further investigated by extending polygenic scoring to incident VTE events, where similar magnitudes of effect were observed for the PRSVTE and the F5 p.R506Q/F2 G20210A mutations. The data indicate that extending current thrombophilia genetic panels to include testing for polygenic VTE risk would significantly increase the yield of current genetic testing and can be warranted.

In conclusion, the data provide new mechanistic insights into the genetic epidemiology of VTE and indicate a greater intersection between blood lipids, VTE, and arterial vascular disease than previously understood.

Those skilled in the art will recognize, or be able to ascertain using no more than routine experimentation, many equivalents to the specific embodiments of the method and compositions described herein. Such equivalents are intended to be encompassed by the following claims.

1. A method of treating venous thromboembolism (VTE) in a subject, the method comprising:
administering a venous thrombus size lowering therapy to the subject, wherein the venous thrombus size lowering therapy is a plasminogen activator inhibitor 1 (PAI-1) inhibitor.

2. The method of claim 1, wherein the PAI-1 inhibitor reduces venous thrombus size in the subject by increasing the conversion of plasminogen to plasmin and increasing the interaction of circulating monocytes with the glycoprotein vitronectin within the thrombus and adjacent vein wall, thereby increasing thrombus clearance and reducing the risk of VTE in the subject.

3. The method of claim 1, wherein the PAI-1 inhibitor is vorapaxar.

4. The method of claim 1, wherein the subject has been determined to be at a greater risk of developing VTE.

5. The method of claim 1, wherein the subject has been identified to have a polygenic risk score that corresponds to a high risk group.

6. The method of claim 1, wherein the subject was determined to be at risk for VTE by identifying the presence of F5 Leidan pR506Q and a prothrombin G20210A SNP in a biological sample from a subject.

7. The method of claim 1, wherein the subject is human.

8. The method of claim 1, further comprising administering a second therapeutic to the subject.

9. The method of claim 8, wherein the second therapeutic is an LDL lowering therapeutic.

10. The method of claim 9, wherein the LDL lowering therapeutic is a statin or a PCSK9 inhibitor.

11. A method of diagnosing and treating venous thromboembolism (VTE), the method comprising:
diagnosing the subject as being at greater risk of developing VTE; and
administering a venous thrombus size lowering therapy to the subject, wherein the venous thrombus size lowering therapy is a plasminogen activator inhibitor (PAI-1) inhibitor.

12. The method of claim 11, further comprising administering a second therapy to the subject based on the diagnosis.

13. The method of claim 12, wherein the second therapy is an LDL lowering therapy.

14. The method of claim 13, wherein the LDL lowering therapy is a statin or a PCSK9 inhibitor.

15. The method of claim 11, wherein diagnosing the subject as being at greater risk of developing VTE further comprises:
identifying whether a F5 Leiden pR506Q or a prothrombin G202010A single nucleotide polymorphism (SNP) are present in a biological sample from the subject.

16. The method of claim 11, wherein diagnosing the subject as being at greater risk of developing VTE further comprises:
identifying the presence of one or more of the 297 SNPs shown in FIG. 19 as present in a biological sample from the subject.

17. The method of claim 11, wherein diagnosing the subject as being at greater risk of VTE further comprises calculating a polygenic risk score (PRS) based on the presence or absence of one or more of the 297 SNPs shown in FIG. 19, wherein the PRS is determined by summing a weighted risk value for each SNP.

18. The method of claim 17, wherein the subject has been identified to have a PRS that corresponds to a high-risk group.

19. The method of claim 18, wherein a PRS within the top 5% of the population indicates the subject is at greater risk of developing VTE.

20. The method of claim 11, wherein the biological sample is blood, plasma, or serum.

21. The method of claim 11, wherein the subject is a human.

22. A method of determining a polygenic risk score (PRS) for developing VTE in a subject comprising:
a. identifying the presence of one or more of the 297 SNPs identified in FIG. 19 are present in a biological sample from the subject; and
b. calculating the PRS by summing the weighted risk score associated with each SNP identified.

23. The method of claim 22, further comprising identifying F5 Leidan pR506Q and prothrombin G20210A single nucleotide polymorphisms in the biological sample from the subject.

24. The method of claim 22, wherein a high PRS indicates a higher risk for developing VTE.

25. The method of claim 22, wherein the PRS is calculated by summing the weighted risk score associated with each SNP identified.

26. The method of claim 22, further comprising diagnosing the subject at risk of developing VTE based on the PRS, and administering a venous thrombus size lowering therapy to the subject based on the diagnosis.
